(19) United States
(12) Patent Application Publication
(10) Pub. No.: US 2026/0151766 A1
(43) Pub. Date: Jun. 4, 2026

(54) LIQUID DELIVERY DEVICE

(71) Applicants: TOKAI UNIVERSITY EDUCATIONAL SYSTEM, Tokyo (JP); SUMITOMO BAKELITE CO., LTD., Tokyo (JP)

(72) Inventors: Hiroshi KIMURA, Kanagawa (JP); Kenta SHINHA, Kanagawa (JP); Shin KOMIYAMA, Kanagawa (JP); Yuki UEDA, Kanagawa (JP); Sohei FUNAOKA, Tokyo (JP); Takuro YOSHIKUNI, Tokyo (JP); Taichi AIHARA, Tokyo (JP)

(21) Appl. No.: 19/122,881

(22) PCT Filed: Oct. 31, 2023
(86) PCT No.: PCT/JP2023/039348

§ 371 Date: Apr. 21, 2025

(30) Foreign Application Priority Data
Oct. 31, 2022 (JP) 2022-174837

(51) Int. Cl. B01L 3/00

(52) U.S. Cl. CPC B01L 3/50273; B01L 2300/0809; B01L 2300/0877

(57) ABSTRACT

A liquid delivery device comprises a liquid delivery unit and a liquid delivery rotary unit. The liquid delivery unit comprises a liquid delivery chamber where liquid flows in and flows out, and first and second straight flow channels. The liquid delivery rotary unit comprises a spindle that protrudes at the center of the liquid delivery chamber, and an impeller, supported by an annular part, to be rotatable about the spindle and contains a magnetic material; a drive motor is disposed outside the liquid delivery chamber and causes the impeller to be rotated by magnetic fields. A gap is provided between the outer circumference of the spindle and the inner circle of the annular part. The impeller rotates in a state where the gap is biased relative to a range of contact between the outer circumference of the spindle and the inner circle of the annular part.

### TECHNICAL FIELD

The present invention relates to a liquid delivery device.

### BACKGROUND ART

In recent years, Microphysiological Systems (hereinafter referred to as MPS) have attracted attention in the field of drug discovery. MPS is a cell assay platform that reproduces the in-vivo environment in vitro. Conventionally, liquid delivery devices have been used for MPS. A liquid delivery device used for MPS is proposed in Patent Literature 1, for example.

The liquid delivery device described in Patent Literature 1 has a liquid flow section through which a liquid flows, and a liquid delivery section that delivers the liquid to the liquid flow section; these sections are connected by a loop-shaped flow path. In this liquid delivery device, the liquid delivery section has a liquid delivery chamber into which the liquid is introduced. The loop-shaped flow path has a first flow path that connects the liquid delivery chamber and the liquid flow section so as to allow liquid delivery, and a second flow path that connects the liquid delivery chamber and the liquid flow section so as to allow liquid delivery. A rotor (impeller) is provided in the liquid delivery chamber to deliver the liquid from one of the first flow path and the second flow path to the other by rotation. The liquid flow section includes one or more reservoirs capable of storing the liquid.

The liquid delivery chamber has two liquid inlets/outlets. One liquid inlet/outlet is located at a rotationally symmetrical position with respect to the other liquid inlet/outlet about the central axis of the liquid delivery chamber. In the liquid delivery device described in Patent Literature 1, the central axis of the liquid delivery chamber is also the central axis of the support shaft and the central axis of the rotor. In other words, one liquid inlet/outlet is located at a position shifted by 180° from the other liquid inlet/outlet in the circumferential direction around the central axis. In still other words, the two liquid inlets/outlets are provided at point-symmetrical positions with respect to the central axis of the liquid delivery chamber.

Further, a convex rotary shaft (support portion) that rotatably supports the rotor (impeller) is formed to project from the inner surface of the liquid delivery chamber; the rotor (impeller) has a bearing portion (annular portion) into which the rotary shaft (support portion) is inserted.

### CITATION LIST

### Patent Literature

Patent Literature 1: JP2021-159008A

### SUMMARY OF INVENTION

### Technical Problem

The present inventors have been further researching and developing the liquid delivery device described in Patent Literature 1, and have found that the direction of liquid flow may reverse, or reverse again and return to the original flow direction, so that the direction of liquid flow is not constant, and a phenomenon of unstable flow rate may occur (refer to [0074], [0075], and the description of FIG. 10 described later). It has been confirmed that such a phenomenon occurs frequently, and it has become clear that this phenomenon causes variations in the liquid delivery state with time. From the viewpoint of performing stable liquid delivery, that is, from the viewpoint of continuously performing liquid delivery in which the direction of liquid flow is constant and the flow rate is also stable, improvement of such a phenomenon is strongly desired.

At present, the cause of this phenomenon is not clear, but the present inventors focused on the gap between the support portion of the liquid delivery chamber that rotatably supports the impeller, and the annular portion of the impeller inserted into the support portion as one of the causes of this phenomenon (this will be described in detail later with reference to FIG. 6A). Although this gap is provided so that the impeller can rotate smoothly, due to the presence of this gap, the contact point between the support portion and the annular portion changes each time the impeller rotates. Therefore, the rotation center of the impeller changes circularly around the center of the support portion each time the impeller rotates. The present inventors have found that when the rotation center of the impeller changes, the position of the blade portion of the impeller also changes, and this affects the state of the liquid being moved by the rotation of the blade portion (this will be described in detail later with reference to FIG. 7A). Since the liquid delivery device is premised on cell culture, it is disposed of after appropriate treatment after use without being reused for hygiene management. Therefore, the liquid delivery device is a consumable item, and it is assumed that it can be mass-produced, so that processing that eliminates the gap by providing a bearing or the like in the gap between the support portion of the liquid delivery chamber that rotatably supports the impeller and the annular portion of the impeller inserted into the support portion to smooth the rotation is not suitable. Moreover, even if the gap is designed to be minimized, it is necessary to adjust the gap to 0.1 mm or less, which is difficult to achieve in mass production.

The present invention has been made in view of the above circumstances. An object of the present invention is to provide a liquid delivery device capable of continuously delivering liquid in a constant flow direction with a stable flow rate.

### Solution to Problem

A liquid delivery device according to the present invention to solve the problem is a liquid delivery device including a liquid delivery section and a liquid delivery rotation section, in which the liquid delivery section includes: a liquid delivery chamber in which liquid flows in and out; and a linear first flow path and a linear second flow path that allow the liquid to flow to and from the liquid delivery chamber in top-down view of the liquid delivery chamber, the liquid delivery rotation section includes: a support shaft that is arranged to project in a center of the liquid delivery chamber; an impeller including an annular portion rotatably supported by the support shaft, and a blade portion provided in the annular portion and causing the liquid in the liquid delivery chamber to flow out of the first flow path or the second flow path, the impeller being made of a material containing a magnetic material; and a drive motor that is arranged outside the liquid delivery chamber and rotates the impeller by a magnetic field, a support portion configured by an outer periphery of the support shaft and an inner circle of the annular portion has a gap for the annular portion to be rotatable, and the drive motor is arranged so that the impeller rotates with the gap being biased in a contact range between the outer periphery of the support shaft and the inner circle of the annular portion.

Preferably, in the top-down view, the first flow path and the second flow path are in any one positional relationship of line symmetry, asymmetry, and point symmetry with respect to the liquid delivery chamber with reference to the support shaft.

Preferably, the liquid delivery chamber is circular in the top-down view, a length of a first wall portion, which is formed by a first connection portion connected to the first flow path, a second connection portion connected to the second flow path, and a circular inner peripheral surface between the first connection portion and the second connection portion in the top-down view of the liquid delivery chamber is shorter than a length of a second wall portion, which is formed by a third connection portion connected to the first flow path, a fourth connection portion connected to the second flow path, and a circular inner peripheral surface between the third connection portion and the fourth connection portion, the first flow path and the second flow path are provided so that an inflow direction of the liquid flowing into the liquid delivery chamber and an outflow direction of the liquid flowing out of the liquid delivery chamber are aligned continuously by an arc of the first wall portion, and the drive motor is arranged so that an area is bisected by a second reference line passing through a center point of the support shaft and orthogonal to a first reference line passing through the center point and a point bisecting a length of the first wall portion, a rotation center of the impeller is located in a semicircular area including the first wall portion, and the impeller rotates with the gap being biased.

Preferably, the liquid delivery chamber is circular in the top-down view, a length of a first wall portion, which is formed by a first connection portion connected to the first flow path, a second connection portion connected to the second flow path, and a circular inner peripheral surface between the first connection portion and the second connection portion in the top-down view of the liquid delivery chamber is shorter than a length of a second wall portion, which is formed by a third connection portion connected to the first flow path, a fourth connection portion connected to the second flow path, and a circular inner peripheral surface between the third connection portion and the fourth connection portion, the first flow path and the second flow path are formed on the same straight line via the liquid delivery chamber, in a concentric circle centered on a center point of the support shaft, the liquid delivery chamber includes the first flow path at a position corresponding to a first quadrant with respect to a third reference line passing through the center point and parallel to an inflow direction of the liquid to the liquid delivery chamber and a fourth reference line orthogonal to the third reference line and passing through the center point, and includes the second flow path at a position corresponding to a second quadrant with respect to the third reference line and the fourth reference line, and the drive motor is arranged so that a rotation center of the impeller is located in an area corresponding to the first quadrant or an area corresponding to a third quadrant with respect to the third reference line and the fourth reference line, and the impeller rotates with the gap being biased.

Preferably, the liquid delivery chamber is circular in the top-down view, a length of a first wall portion, which is formed by a first connection portion connected to the first flow path, a second connection portion connected to the second flow path, and a circular inner peripheral surface between the first connection portion and the second connection portion in the top-down view of the liquid delivery chamber is shorter than a length of a second wall portion, which is formed by a third connection portion connected to the first flow path, a fourth connection portion connected to the second flow path, and a circular inner peripheral surface between the third connection portion and the fourth connection portion, a formation direction of the first flow path with respect to the liquid delivery chamber and a formation direction of the second flow path with respect to the liquid delivery chamber are parallel, but the first flow path and the second flow path are formed to be offset with respect to each other via the liquid delivery chamber, the impeller rotates in the same direction as an outflow direction of the liquid flowing from the first flow path to the second flow path, in a concentric circle centered on a center point of the support shaft, the liquid delivery chamber includes the first flow path at a position including a boundary line between a first quadrant and a fourth quadrant with respect to a third reference line passing through the center point and parallel to an inflow direction of the liquid to the liquid delivery chamber and a fourth reference line orthogonal to the third reference line and passing through the center point, and includes the second flow path at a position corresponding to a second quadrant with respect to the third reference line and the fourth reference line, and the drive motor is arranged so that a rotation center of the impeller is located in an area corresponding to the first quadrant or an area corresponding to a third quadrant with respect to the third reference line and the fourth reference line, and the impeller rotates with the gap being biased.

Preferably, the liquid delivery chamber is circular in the top-down view, a length of a first wall portion, which is formed by a first connection portion connected to the first flow path, a second connection portion connected to the second flow path, and a circular inner peripheral surface between the first connection portion and the second connection portion in the top-down view of the liquid delivery chamber is shorter than a length of a second wall portion, which is formed by a third connection portion connected to the first flow path, a fourth connection portion connected to the second flow path, and a circular inner peripheral surface between the third connection portion and the fourth connection portion, a formation direction of the first flow path with respect to the liquid delivery chamber and a formation direction of the second flow path with respect to the liquid delivery chamber are parallel, but the first flow path and the second flow path are formed to be offset with respect to each other via the liquid delivery chamber, the impeller rotates in the same direction as an outflow direction of the liquid flowing from the second flow path to the first flow path, in a concentric circle centered on a center point of the support shaft, the liquid delivery chamber includes the first flow path at a position including a boundary line between a first quadrant and a fourth quadrant with respect to a third reference line passing through the center point and parallel to an inflow direction of the liquid to the liquid delivery chamber and a fourth reference line orthogonal to the third reference line and passing through the center point, and includes the second flow path at a position corresponding to a second quadrant with respect to the third reference line and the fourth reference line, and the drive motor is arranged so that a rotation center of the impeller is located in an area corresponding to the first quadrant, an area corresponding to a second quadrant, or an area corresponding to a fourth quadrant, and the impeller rotates with the gap being biased.

Preferably, the liquid delivery chamber is circular in the top-down view, the liquid delivery chamber has a first wall portion and a second wall portion, the first wall portion is formed by a first connection portion connected to the first flow path, a second connection portion connected to the second flow path, and a first inner peripheral surface forming a circular arc between the first connection portion and the second connection portion in the top-down view, the second wall portion is formed by a third connection portion connected to the first flow path, a fourth connection portion connected to the second flow path, a second inner peripheral surface forming a circular arc between the third connection portion and the fourth connection portion, a third inner peripheral surface having an arc shape and a curvature opposite to that of the second inner peripheral surface between the third connection portion and the second inner peripheral surface, and a fourth inner peripheral surface having an arc shape and a curvature opposite to that of the second inner peripheral surface between the fourth connection portion and the second inner peripheral surface, the first flow path and the second flow path are provided so that an inflow direction of the liquid flowing into the liquid delivery chamber and an outflow direction of the liquid flowing out of the liquid delivery chamber are aligned continuously by an arc of the first wall portion, and the drive motor is arranged so that an area is bisected by a second reference line passing through a center point of the support shaft and orthogonal to a first reference line passing through the center point and a point bisecting a length of the first wall portion, a rotation center of the impeller is located in a semicircular area including the first wall portion, and the impeller rotates with the gap being biased.

Preferably, taking, as a reference, a fifth reference line that passes through an intersection between the second inner peripheral surface and the first reference line and is parallel to the second reference line, formation dimensions of the third inner peripheral surface and the fourth inner peripheral surface, measured perpendicular to the fifth reference line at longest points thereof, are both the same.

Preferably, taking, as a reference, a fifth reference line that passes through an intersection between the second inner peripheral surface and the first reference line and is parallel to the second reference line, formation dimensions of the third inner peripheral surface and the fourth inner peripheral surface, measured perpendicular to the fifth reference line at longest points thereof, are different from each other.

Preferably, in the liquid delivery chamber, when the liquid is delivered by rotation of the impeller, a flow rate of the liquid is adjusted by a rotation speed of the impeller.

Preferably, a liquid delivery direction of the liquid is adjusted by a rotation direction of the impeller.

Preferably, the liquid delivery device further includes: a first reservoir communicating with the first flow path; a second reservoir communicating with the second flow path; and a return flow path connected to the first reservoir and the second reservoir, the first flow path, the second flow path, and the return flow path form a loop-shaped flow path, a depth dimension of the loop-shaped flow path and a depth dimension of the liquid delivery chamber are the same, and a flow rate of the liquid is adjusted by the depth dimension.

Preferably, a flow rate of the liquid is adjusted by a width dimension of the return flow path.

### Advantageous Effects of Invention

The liquid delivery device according to the present invention can continuously deliver liquid in a constant flow direction with a stable flow rate.

### BRIEF DESCRIPTION OF DRAWINGS

FIG. 1 is a perspective view showing an example of the liquid delivery device according to the overall configuration based on the liquid delivery section of the first embodiment.

FIG. 2 is an exploded perspective view showing an example of the liquid delivery device according to the overall configuration based on the liquid delivery section of the first embodiment.

FIG. 3 is a plan view showing an example of the device body according to the overall configuration based on the liquid delivery section of the first embodiment.

FIG. 4 is a perspective view showing a part of an example of the device body according to the overall configuration based on the liquid delivery section of the first embodiment.

FIG. 5 is a configuration diagram of the liquid delivery section and the impeller based on the liquid delivery section of the first embodiment.

FIGS. 6A and 6B are each an explanatory diagram showing a difference in the rotation center position of the impeller between the conventional method and the present embodiment (point contact method) with respect to the first embodiment. FIG. 6A is an explanatory diagram showing the relationship between the central axis of the support shaft and the rotation center of the impeller in the conventional method. FIG. 6B is an explanatory diagram showing the relationship between the center point of the support shaft and the rotation center of the impeller in the point contact method.

FIGS. 7A and 7B are each an explanatory diagram showing a difference in the rotation center position of the impeller between the conventional method and the present embodiment (point contact method) with respect to the first embodiment. FIG. 7A is an explanatory diagram showing the degree of variation in the rotation center of the impeller in the conventional method. FIG. 7B is an explanatory diagram showing the degree of variation in the rotation center of the impeller in the point contact method.

FIG. 8 is an explanatory diagram showing a difference in the rotation center position of the impeller between the conventional method and the present embodiment (point contact method) with respect to the first embodiment.

FIG. 9 is a graph showing the results of measuring the average flow rate of the impeller in the point contact method over time, which was measured when the investigation regarding FIG. 8 was conducted.

FIG. 10 is a graph showing a backflow phenomenon that occurred when a liquid delivery chamber in which the first flow path and the second flow path are provided point-symmetrically with respect to the support shaft is used in the liquid delivery device.

FIG. 11 is an explanatory diagram of the liquid delivery section of the liquid delivery device according to the second embodiment.

FIG. 12 is an explanatory diagram of the liquid delivery section of the liquid delivery device according to the third embodiment.

FIG. 13 is an explanatory diagram of the liquid delivery section of the liquid delivery device according to the fourth embodiment.

FIGS. 14A to 14D are explanatory diagrams showing the shape of the liquid delivery section of the device body and the arrangement position of the rotation center of the drive motor, which will be described later, according to Examples 1 to 4, respectively.

FIG. 15 is an explanatory diagram showing how the impeller rotates and how the average flow rate is measured in the examples.

FIG. 16 is a graph showing changes over time in the average flow rate when the rotation center of the drive motor is arranged at five locations: the center, upper right, upper left, lower left, and lower right of the liquid delivery chamber (point symmetry) of the liquid delivery device according to Example 1 in FIG. 14A (number of devices used: 4).

FIG. 17 is a graph showing changes over time in the average flow rate when the rotation center of the drive motor is arranged at five locations: the center, upper right, upper left, lower left, and lower right of the liquid delivery chamber (asymmetrical) of the liquid delivery device according to Example 2 in FIG. 14B (number of devices used: 4).

FIG. 18 is a graph showing changes over time in the average flow rate when the rotation center of the drive motor is arranged at five locations: the center, upper right, upper left, lower left, and lower right of the liquid delivery chamber (line symmetry Ver. 1) of the liquid delivery device according to Example 3 in FIG. 14C (number of devices used: 4).

FIG. 19 is a graph showing changes over time in the average flow rate when the rotation center of the drive motor is arranged at five locations: the top, center, bottom, upper left, and upper right of the liquid delivery chamber (line symmetry Ver. 2) of the liquid delivery device according to Example 4 in FIG. 14D (number of devices used: 3).

FIG. 20 is a graph showing the flow rate, average flow rate, and coefficient of variation of each device used in the liquid delivery devices according to Examples 1 to 4 after 45 minutes from the start of driving the drive motor.

FIG. 21 is a graph showing that the average flow rate of the liquid delivery device has rotation speed dependency.

FIG. 22 is a graph showing that the average flow rate of the liquid delivery device according to Example 4 in FIG. 14D has flow path depth dependency.

FIG. 23 is a graph showing that the average flow rate of the liquid delivery devices according to Example 4 with different average flow path depths in FIG. 14D has rotation speed dependency.

FIG. 24 is an explanatory diagram showing the shape of the liquid delivery section of the device body and the arrangement position of the rotation center of the drive motor according to Example 5.

FIG. 25 is an explanatory diagram of the liquid delivery section of the liquid delivery device according to a modification of the third embodiment.

FIG. 26 is an explanatory diagram of the liquid delivery section of the liquid delivery device according to a modification of the fourth embodiment.

FIG. 27 is an explanatory diagram of the liquid delivery section of the liquid delivery device according to the sixth embodiment.

FIG. 28 is a perspective view seen from the direction of arrow XXVIII in FIG. 27.

FIG. 29 is an explanatory diagram of the liquid delivery section of the liquid delivery device according to a modification of the sixth embodiment.

FIG. 30 is an explanatory diagram of the liquid delivery section of the liquid delivery device according to a modification of the sixth embodiment.

FIG. 31 is an explanatory diagram of the liquid delivery section of the liquid delivery device according to a further modification of the sixth embodiment.

FIG. 32 is an explanatory diagram showing the shapes of the liquid delivery sections according to Nos. 1 to 6 that were adopted when investigating how the difference in the shape of the liquid delivery section of the device body affects the liquid flow rate.

FIG. 33 is a graph showing individual flow rates and average flow rates [μL/min] when Nos. 1 to 6 were measured three times each.

FIG. 34 is an explanatory diagram showing and explaining the conditions, etc., of the simulation for examining the shape of the liquid delivery section of the device body.

FIG. 35 is a graph showing the results of the simulation.

FIG. 36 is an explanatory diagram showing a graph (left graph) showing the average flow rates described in the table shown in FIG. 35 (experimental results in the simulation) in a bar graph, and a graph (right graph) extracting the experimental results of Nos. 1, 3, 4, and 6 (experimental results with actual devices) from the graph shown in FIG. 33.

FIG. 37 is an explanatory diagram showing and explaining the conditions, etc., of the experiment to examine the influence of the width dimension of the return flow path.

FIG. 38 is a graph showing the results of verifying the influence of the width dimension of the return flow path.

### DESCRIPTION OF EMBODIMENTS

Hereinafter, a liquid delivery device according to an embodiment of the present invention will be described with reference to the drawings as appropriate. In all drawings for explaining the present invention, the same reference numerals are given to the same functional elements, and repeated explanations thereof may be omitted.

### [Liquid Delivery Device]

### (Overall Configuration)

First, an example of the overall configuration of the liquid delivery device 100 will be described, and then the specific contents and preferred aspects of the present invention will be described. The overall configuration will be described by taking the liquid delivery section 3 (liquid delivery chamber 7) having a point-symmetrical shape, which will be described in the first embodiment, as an example.

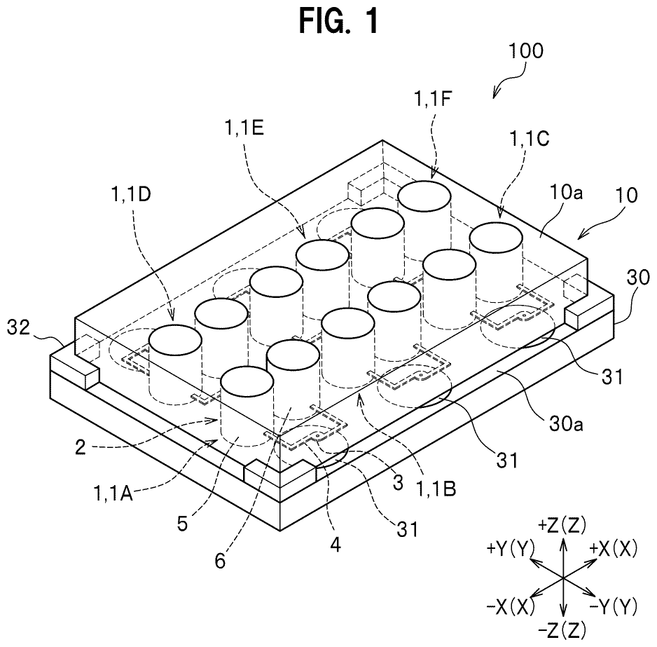

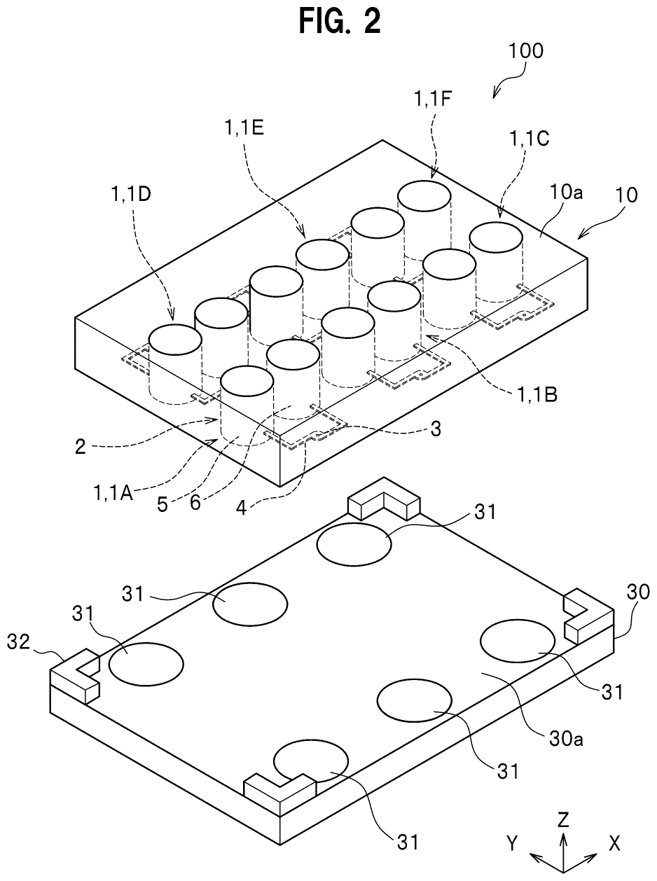

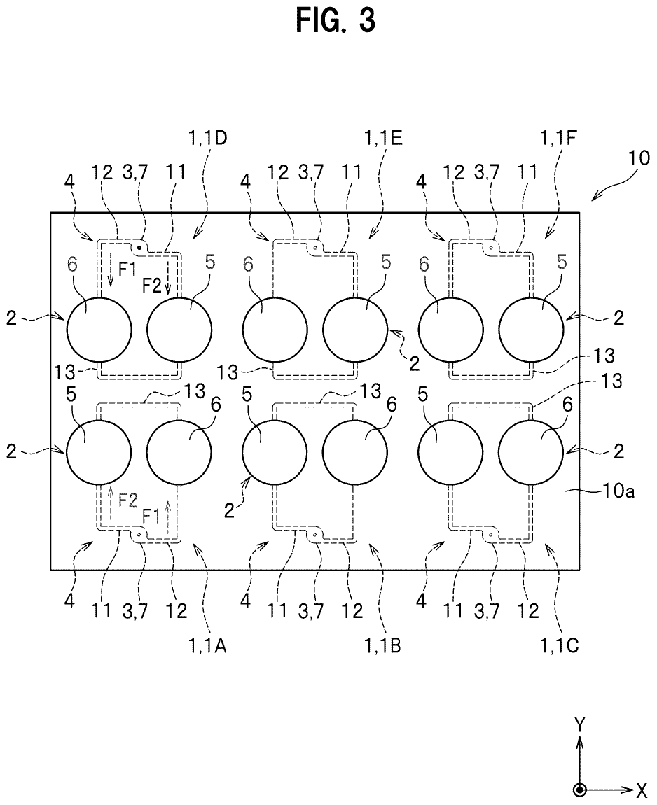

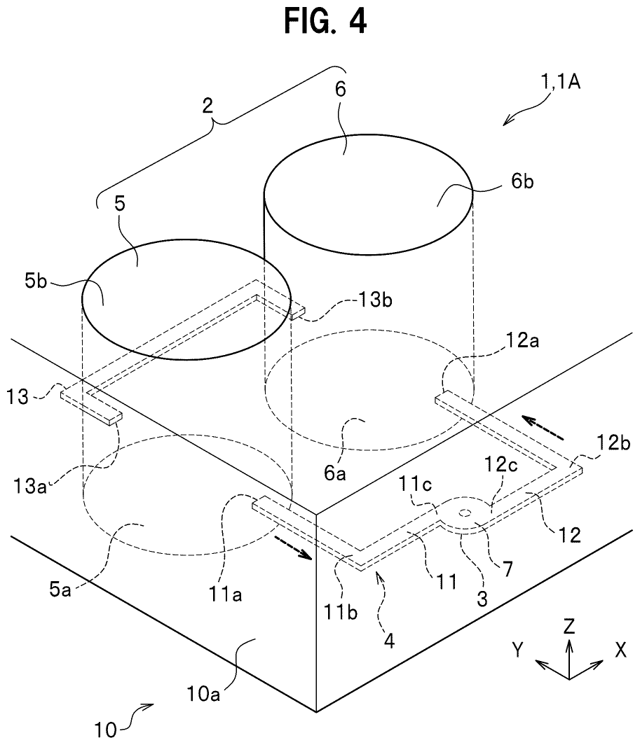

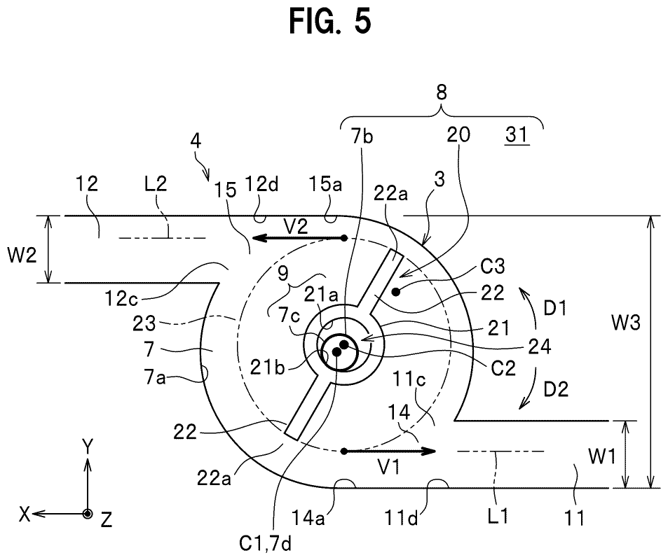

FIG. 1 is a perspective view showing an example of the liquid delivery device 100 according to the overall configuration based on the liquid delivery section 3 of the first embodiment. FIG. 2 is an exploded perspective view showing an example of the liquid delivery device 100 according to the overall configuration based on the liquid delivery section 3 of the first embodiment. FIG. 3 is a plan view showing an example of the device body 10 according to the overall configuration based on the liquid delivery section 3 of the first embodiment. FIG. 4 is a perspective view showing a part of an example of the device body 10 according to the overall configuration based on the liquid delivery section 3 of the first embodiment. FIG. 5 is a configuration diagram of the liquid delivery section 3 and the impeller 20 based on the liquid delivery section 3 of the first embodiment.

Hereinafter, the positional relationship of each component may be described using an XYZ orthogonal coordinate system. The X direction is the longitudinal direction of the rectangular plate-shaped device body 10. The +X direction is the right direction. The −X direction is the left direction. The Y direction is the front-rear direction orthogonal to the X direction. The +Y direction is the rear direction. The −Y direction is the forward direction. The Z direction is the thickness direction of the device body 10 and is orthogonal to the X and Y directions. The +Z direction is the upward direction. The −Z direction is the downward direction. The Z direction is also referred to as the vertical direction or the depth direction. Viewing from the Z direction (vertical direction), that is, viewing in the horizontal direction is called a top-down view, and a drawing using the top-down view is called a plan view.

As shown in FIG. 3, in this embodiment, the direction in which the liquid circulates in the loop-shaped flow path 4 in the liquid delivery device 100 is defined as follows: the counterclockwise direction (left rotation) is the forward flow F1, and the clockwise direction (right rotation) is the reverse flow F2.

Further, as shown in FIG. 5, the impeller 20 is rotatably supported by the support shaft 7b, but in this embodiment, to maintain a consistent direction of liquid flow, the impeller 20 is rotated in the rotation direction D1 (counterclockwise direction) in top-down view (rotated to the left). Hereinafter, in this embodiment, the liquid delivery device 100 will be described by taking as an example a case where the liquid circulates in the loop-shaped flow path 4 in the counterclockwise direction (left rotation), that is, in the forward flow F1 direction.

In this embodiment, the liquid can also be circulated in the clockwise direction (right rotation), that is, in the reverse flow F2 direction. In this case, the impeller 20 may be rotated in the rotation direction D2 (clockwise direction) (may be rotated to the right). In this case, the shape of the liquid delivery section 3, which will be described later, and the like may be a mirror image of the case of the counterclockwise direction (left rotation).

As shown in FIGS. 1 to 3, the liquid delivery device 100 includes a device body 10 and a drive device 30 (see FIGS. 1 and 2). As will be described later, the device body 10 is separable from the drive device 30, and is used by placing the device body 10 on the drive device 30. When the device body 10 is placed on the drive device 30, the liquid delivery section 3 provided in the device body 10 is placed on the drive motor 31 provided in the drive device 30.

The liquid delivery device 100 having such a configuration can be used as, for example, a cell culture device. When the liquid delivery device 100 is used as a cell culture device, for example, cells can be accommodated in the first reservoir 5 and a liquid medium for culturing cells can be accommodated in the second reservoir 6. Then, it can be used by delivering the liquid medium for culturing cells from the second reservoir 6 to the first reservoir 5 via the liquid delivery section 3.

The device body 10 includes a first reservoir 5 and a second reservoir 6. The device body 10 includes a first flow path 11, a liquid delivery section 3 (liquid delivery chamber 7), a second flow path 12, and a return flow path 13. The first reservoir 5 and the second reservoir 6 are connected by the first flow path 11, the liquid delivery section 3 (liquid delivery chamber 7), and the second flow path 12. Further, the first reservoir 5 and the second reservoir 6 are connected by the return flow path 13. As an example, the device body 10 includes multiple first reservoirs 5 and second reservoirs 6. Specific configurations thereof will be described later.

As shown in FIGS. 1 and 2, the drive device 30 includes four positioning portions 32 whose height is one step higher for positioning the device body 10. The positioning portions 32 are provided at the four corners of the drive device 30. Here, each positioning portion 32 has a substantially L-shape in top-down view, and each corner of the device body 10 abuts against the inside of each positioning portion 32. That is, the device body 10 is accurately positioned and stably provided by placing it while bringing its four corners into contact with the positioning portions 32 of the drive device 30. The positioning portions 32 only need to determine the position of the device body 10, and the shape thereof is not limited to an L-shape. For example, the positioning portion 32 may be two columnar protrusions, or may be configured by one or more combinations arbitrarily selected from an L-shape, two columnar protrusions, and one columnar protrusion. The drive device 30 is arranged so that the rotation center C3 (FIG. 5) of the drive motor 31 is accommodated in the liquid delivery section 3 of the liquid delivery device 100, specifically, in the liquid delivery chamber 7. The drive motor 31 is arranged outside the liquid delivery chamber 7 and rotates the impeller 20 by a magnetic field. That is, the drive motor 31 is a magnetic field generating device, and is also called a magnetic stirrer or the like. Details of the arrangement position of the rotation center C3 of the drive motor 31 will be described later. Since the liquid delivery device 100 has such a configuration, the position of the drive motor 31 of the drive device 30 and the position of the liquid delivery chamber 7 can be matched simply by placing the device body 10 while bringing the corners of the four corners thereof into contact with the positioning portions 32 of the drive device 30.

The device body 10 is formed in, for example, a block shape or a plate shape. The device body 10 has a rectangular shape (for example, a rectangular shape) in top-down view. The device body 10 is made of, for example, resin. Examples of the resin constituting the device body 10 include polystyrene resin (PS); polyester resin such as polyethylene terephthalate (PET); acrylic resin such as polymethyl methacrylate resin (PMMA); polyolefin resin such as cycloolefin polymer (COP); polycarbonate resin; and silicone material such as polydimethylsiloxane (PDMS). A transparent material is preferable, but it may be colored.

As shown in FIGS. 1 to 3, the device body 10 may be configured by combining a resin main portion (not shown) in which a liquid flow structure 1 is formed and a bottom member (not shown) such as a plate or a sheet. The bottom member closes the lower opening of the reservoir and the like. The bottom member is not particularly limited in material and the like as long as it can store liquid in the first reservoir 5 and the second reservoir 6. The bottom member may be made of, for example, silicone resin or olefin resin having high gas permeability. According to this configuration, oxygen can be efficiently supplied to the cells cultured on the bottom member, so that the physiological activity of aerobic cells can be improved. In addition, the bottom member may be made of a cover glass. According to this configuration, a confocal microscope can be used to observe the state of the cells in the first reservoir 5 and the second reservoir 6 in detail through the cover glass.

The device body 10 may be configured by one or more cylindrical bodies that form a reservoir inside and a frame body that supports the cylindrical bodies.

As shown in FIGS. 1 to 3, the device body 10 has multiple (for example, six) liquid flow structures 1. The six liquid flow structures 1 (1A to 1F) are arranged in a “3×2” matrix in the X and Y directions. The number of liquid flow structures 1 is not limited to six, and may be one according to the experimental system, or may be designed to be any number of two or more, for example, 12, 24, 48, or 96. The direction in which the multiple liquid flow structures 1 are arranged is not limited to the arrangement in FIGS. 1 to 3. In FIGS. 1 to 3, the liquid delivery sections 3 (liquid delivery chambers 7) are arranged so as to face outward in the +Y direction (rearward) and the −Y direction (forward), respectively, but for example, the liquid flow structures TA, 1B, and 1C may all be arranged in the same direction as the liquid flow structures 1D, 1E, and 1F. Further, each liquid flow structure 1 may be arranged in any direction.

FIG. 4 shows the liquid flow structure TA among the six liquid flow structures 1 (TA to 1F) shown in FIG. 3. As shown in FIG. 4, the liquid flow structure 1 has a liquid flow section 2, a liquid delivery section 3, and a loop-shaped flow path 4.

The liquid flow section 2 includes a first reservoir 5 and a second reservoir 6.

The first reservoir 5 and the second reservoir 6 are recesses formed to open to, for example, the main surface 10a (upper surface) of the device body 10 (see FIGS. 1 and 2). The first reservoir 5 and the second reservoir 6 are, for example, circular in top-down view. The first reservoir 5 and the second reservoir 6 have a columnar internal space (reservoir space) having a central axis along the Z direction. Therefore, the first reservoir 5 has a bottom surface 5a and an inner wall surface 5b, and the second reservoir 6 has a bottom surface 6a and an inner wall surface 6b. The first reservoir 5 and the second reservoir 6 are formed side by side in the X direction.

The first reservoir 5 and the second reservoir 6 have upper openings, thereby serving as open reservoirs. Therefore, a user can easily perform operations such as cell seeding and medium exchange on the first reservoir 5 and the second reservoir 6 through the upper openings. Since the first reservoir 5 and the second reservoir 6 are open reservoirs, operations for maintaining airtightness and the like are not required, and the setup of the liquid delivery device 100 is easy.

When the liquid delivery device 100 is used as a cell culture device, the liquid delivery device 100 may include a structure (cell culture section) capable of cell culture in the liquid flow structure 1. Examples of the cell culture section include a cell culture insert. Cells may be accommodated in the cell culture insert, and the cell culture insert may be set in the first reservoir 5. Specifically, cells may be attached to the porous membrane at the bottom of the cup-type cell culture insert, and the cell culture insert to which the cells are attached may be set so as to be immersed in the liquid medium in the first reservoir 5. Similarly, a flat culture substrate such as CELL DESK that is not cup-type may be used in the same manner.

The liquid delivery section 3 has a liquid delivery chamber 7.

The loop-shaped flow path 4 has a first flow path 11, a second flow path 12, and a return flow path 13. The loop-shaped flow path 4 connects the liquid flow section 2 and the liquid delivery section 3. That is, the loop-shaped flow path 4 connects the first reservoir 5, the second reservoir 6, and the liquid delivery chamber 7. Further, as described above, the return flow path 13 is connected to the first reservoir 5 and the second reservoir 6. The first flow path 11, the second flow path 12, and the return flow path 13 form a circulation flow path.

In the present embodiment, the loop-shaped flow path 4 formed by the first flow path 11, the second flow path 12, and the return flow path 13, and the liquid delivery chamber 7 preferably have the same depth dimension. In this embodiment, the liquid flow rate can be adjusted by this depth dimension. That is, by increasing this depth dimension, the liquid delivery device 100 with a large liquid flow rate can be realized, and by decreasing this depth dimension, the liquid delivery device 100 with a small liquid flow rate can be realized.

Further, in this embodiment, the liquid flow rate can also be adjusted by the width dimension of the return flow path 13. That is, the larger the width dimension of the return flow path 13, the larger the liquid flow rate.

One end 11a of the first flow path 11 communicates with the inner wall surface 5b of the first reservoir 5. The first flow path 11 extends in the −Y direction from the one end 11a, bends at a bent portion 11b, and extends in the +X direction. A connection end 11c, which is the other end of the first flow path 11, is connected to the liquid delivery chamber 7. As described above, the depth dimension of the first flow path 11 is preferably the same as that of the second flow path 12 and the like, but may not be the same. The cross-sectional shape of the first flow path 11 (the shape of across section orthogonal to the length direction of the first flow path 11) is not particularly limited as long as the liquid can circulate, and may be, for example, a rectangle, a polygon, a circle, an ellipse, or a semi-cylinder.

One end 12a of the second flow path 12 communicates with the inner wall surface 6b of the second reservoir 6. The second flow path 12 extends in the −Y direction from the one end 12a, bends at a bent portion 12b, and extends in the −X direction. A connection end 12c, which is the other end of the second flow path 12, is connected to the liquid delivery chamber 7. As described above, the depth dimension of the second flow path 12 is preferably the same as that of the first flow path 11 and the like, but may not be the same. The cross-sectional shape of the second flow path 12 (the shape of a cross section orthogonal to the length direction of the second flow path 12) is not particularly limited as long as the liquid can circulate, and may be, for example, a rectangle, a polygon, a circle, an ellipse, or a semi-cylinder.

The cross-sectional shape of the return flow path 13 (the shape of a cross section orthogonal to the length direction of the return flow path 13) is not particularly limited as long as the liquid can circulate, and may be, for example, a rectangle, a polygon, a circle, an ellipse, or a semi-cylinder.

As shown in FIG. 5, the liquid delivery device 100 includes a liquid delivery section 3 and a liquid delivery rotation section 8. The liquid delivery section 3 includes a liquid delivery chamber 7, a first flow path 11, and a second flow path 12. The liquid delivery rotation section 8 includes a support shaft 7b, an impeller 20, and a drive motor 31.

The liquid delivery chamber 7 allows liquid to flow in and out. The first flow path 11 and the second flow path 12 allow the liquid to flow to and from the liquid delivery chamber 7.

The first flow path 11 has a linear shape (straight pipe shape) at a connection portion with the liquid delivery chamber 7.

The first flow path 11 continuously allows the flow of liquid into or out of the liquid delivery chamber 7 on one side of the liquid delivery chamber 7 in top-down view.

The second flow path 12 also has a linear shape (straight pipe shape) at a connection portion with the liquid delivery chamber 7.

The second flow path 12 continuously allows the flow of liquid into or out of the liquid delivery chamber 7 on the other side of the liquid delivery chamber 7 in the top-down view.

In the flow path in which the liquid flows in a certain direction, any of the first flow path 11 and the second flow path 12 may be an inflow path or an outflow path. Which of these is the inflow path or the outflow path can be adjusted by the rotation direction of the impeller 20 as described above.

The support shaft 7b is arranged to project in the center of the liquid delivery chamber 7. The impeller 20 has an annular portion 21 that is rotatably supported by the support shaft 7b. Further, the impeller 20 has a blade portion 22 that is provided in the annular portion 21 and causes the liquid in the liquid delivery chamber 7 to flow out of the first flow path 11 or the second flow path 12 (as described above, in the example of this embodiment, it causes the liquid to flow out of the second flow path 12). Further, the impeller 20 is made of a material containing a magnetic material. The impeller 20 is also called a rotor, a stirrer, a magnetic stirrer bar, or the like. The drive motor 31 is arranged outside the liquid delivery chamber 7 and rotates the impeller 20 by a magnetic field.

As shown in FIG. 5, in the liquid delivery device 100, a support portion 9 configured by the outer periphery 7c of the support shaft 7b and the inner circle 21a of the annular portion 21 has a gap 24 for the annular portion 21 to be rotatable. Further, in the liquid delivery device 100, the drive motor 31 described above is arranged so that the impeller 20 can rotate with the gap 24 being biased in the contact range between the outer periphery 7c of the support shaft 7b and the inner circle 21a of the annular portion 21.

As shown in FIG. 5, the width W1 of the first flow path 11 and the width W2 of the second flow path 12 are preferably equal to or less than half the width W3 of the liquid delivery chamber 7. The width W3 of the liquid delivery chamber 7 is the inner diameter of the liquid delivery chamber 7.

The first flow path 11 and the second flow path 12 may have the same flow resistance, but may have different flow resistances. However, it is desirable that the first flow path 11 and the second flow path 12 have the same flow resistance. The flow resistance is a value depending on the cross-sectional area and length of the flow path. As the cross-sectional area of the flow path increases, the flow resistance decreases. Further, as the length of the flow path decreases, the flow resistance decreases.

In a case where the first flow path 11 and the second flow path 12 have different flow resistance configurations, for example, when the liquid is delivered in the forward flow F1 direction, the flow resistance of the second flow path 12 is preferably smaller than that of the first flow path 11. By doing so, the liquid can be smoothly discharged from the liquid delivery chamber 7 to the second flow path 12. When the liquid is delivered in the reverse flow F2 direction, the flow resistance of the first flow path 11 is preferably smaller than that of the second flow path 12. By doing so, the liquid can be smoothly discharged from the liquid delivery chamber 7 to the first flow path 11.

### First Embodiment

As shown in FIG. 5, the liquid delivery chamber 7 is circular in top-down view. The liquid delivery chamber 7 has an inner diameter (width W3) in top-down view such that the tip 22a of the impeller 20 does not abut against the inner peripheral surface 7a of the liquid delivery chamber 7 even if the rotation trajectory 23 of the impeller 20 is biased. The liquid delivery chamber 7 has a cylindrical internal space having a central axis C1 along the Z direction. The shape of the internal space of the liquid delivery chamber 7 may be a truncated cone shape or a disk shape. In this embodiment, the support shaft 7b is formed to project in the center of the liquid delivery chamber 7 along the central axis C1. Therefore, the central axis C1 of the liquid delivery chamber 7 is also the central axis passing through the center point 7d of the support shaft 7b. The support shaft 7b is formed in, for example, a cylindrical shape or a conical shape. The support shaft 7b rotatably supports the impeller 20.

The liquid delivery chamber 7 has a liquid inlet/outlet 14 and a liquid inlet/outlet 15. In this embodiment, since the impeller 20 of the liquid delivery chamber 7 rotates in the rotation direction D1 (counterclockwise direction) and allows the flow of the liquid in the forward flow F1 direction, the liquid inlet/outlet 14 serves as an inlet, and the liquid inlet/outlet 15 serves as an outlet.

As described above, when the impeller 20 of the liquid delivery chamber 7 rotates in the rotation direction D2 (clockwise direction) and allows the flow of the liquid in the reverse flow F2 direction, the liquid inlet/outlet 14 serves as an outlet, and the liquid inlet/outlet 15 serves as an inlet.

The liquid inlet/outlet 14 is formed in the inner peripheral surface 7a of the liquid delivery chamber 7. The liquid delivery chamber 7 communicates with the first flow path 11 through the liquid inlet/outlet 14. The liquid delivery chamber 7 is connected to the first flow path 11 so that the liquid can flow through the liquid inlet/outlet 14. The inner side surface 11d of the linear first flow path 11 in top-down view and the arc-shaped inner peripheral surface 7a are formed to be smoothly continuous. An end 14a of the liquid inlet/outlet 14 is a connection point between the inner side surface 11d of the first flow path 11 and the inner peripheral surface 7a.

In top-down view, the first flow path 11 in the length range including the connection end 11c extends in the traveling direction of the tip 22a of the blade portion 22 of the impeller 20 (the direction of the velocity vector V1) starting from the same circumferential position as the end 14a. The direction of the first flow path 11 in the length range including the connection end 11c is the direction along the tangent L1 of the rotation trajectory 23 at the same circumferential position as the end 14a.

The liquid inlet/outlet 15 is formed in the inner peripheral surface 7a of the liquid delivery chamber 7. The liquid delivery chamber 7 communicates with the second flow path 12 through the liquid inlet/outlet 15. The liquid delivery chamber 7 is connected to the second flow path 12 so that the liquid can flow through the liquid inlet/outlet 15. The inner side surface 12d of the linear second flow path 12 in top-down view and the arc-shaped inner peripheral surface 7a are formed to be smoothly continuous. An end 15a of the liquid inlet/outlet 15 is a connection point between the inner side surface 12d of the second flow path 12 and the inner peripheral surface 7a.

The liquid inlet/outlet 15 is located at a rotationally symmetrical (point-symmetrical) position with respect to the liquid inlet/outlet 14 with respect to the support shaft 7b of the liquid delivery chamber 7, more specifically, the central axis C1 of the liquid delivery chamber 7. That is, the liquid inlet/outlet 15 is located at a position shifted by 180° from the liquid inlet/outlet 14 in the circumferential direction around the support shaft 7b. The liquid inlet/outlet 15 only needs to be located at a position where at least a part of the circumferential direction is rotationally symmetrical with the liquid inlet/outlet 14. That is, the liquid inlet/outlet 14 and the liquid inlet/outlet 15 only need to be located at positions where at least a part thereof in the circumferential direction is shifted by 180° in the circumferential direction around the central axis C1.

In top-down view, the second flow path 12 in the length range including the connection end 12c extends in the traveling direction of the tip 22a of the blade portion 22 of the impeller 20 (the direction of the velocity vector V2) starting from the same circumferential position as the end 15a. The direction of the second flow path 12 in the length range including the connection end 12c is the direction along the tangent L2 of the rotation trajectory 23 at the same circumferential position as the end 15a.

As shown in FIGS. 3 and 4, the return flow path 13 connects the first reservoir 5 and the second reservoir 6. One end 13a of the return flow path 13 is connected to the inner wall surface 5b of the first reservoir 5. The other end 13b of the return flow path 13 is connected to the inner wall surface 6b of the second reservoir 6. In the liquid delivery device 100, since the liquid flows counterclockwise in the liquid flow structure 1, the liquid in the second reservoir 6 flows toward the first reservoir 5 through the return flow path 13.

The liquid flow structure 1 (the liquid flow section 2, the liquid delivery section 3, and the loop-shaped flow path 4) can be formed by microfabrication techniques such as a three-dimensional plotter, a three-dimensional printer, or photolithography.

As shown in FIG. 5, the impeller 20 includes an annular portion 21 and multiple (for example, two) blade portions 22. Part or all of the impeller 20 is made of a magnetic material. The impeller 20 may include a permanent magnet. A resin coating layer excellent in wear resistance may be formed on the surface of the impeller 20. The resin coating layer is made of, for example, fluororesin.

The shape of the annular portion 21 may be cylindrical. The annular portion 21 is rotatably supported by the support shaft 7b. Therefore, the inside (inner circle 21a) of the annular portion 21 functions as a bearing portion 21b into which the support shaft 7b is inserted. In the liquid delivery device 100 according to this embodiment, the rotation center C2 of the annular portion 21, that is, the rotation center C2 of the impeller 20 does not coincide with the central axis C1 of the liquid delivery chamber 7 by adopting the configuration described later.

The blade portions 22 are formed in a flat plate shape or a rod shape and extend radially outward from the outer peripheral surface of the annular portion 21. The two blade portions 22, 22 are formed at rotationally symmetrical positions with respect to the rotation center C2 of the impeller 20. The two blade portions 22, 22 have the same extension length from the annular portion 21. The number of blade portions 22 is not limited to two, and may be any number of three or more. The rotation trajectory 23 is a circular rotation trajectory drawn by the tips 22a of the blade portions 22 when the impeller 20 rotates.

As shown in FIG. 2, the drive device 30 has a rectangular mounting surface 30a in top-down view. The device body 10 is placed on the mounting surface 30a.

The drive device 30 includes a drive motor 31 at a position overlapping the liquid delivery chamber 7 of each liquid flow structure 1 (1A to 1F). The drive motor 31 includes a rotating magnet (not shown). This rotating magnet is rotated by a drive source such as an electric motor. A rotation center C3 of the drive motor 31 is generated as the rotating magnet of the drive motor 31 rotates. Further, due to a change in the magnetic field accompanying the rotation of the rotating magnet of the drive motor 31, a rotational driving force around the rotation center C2 is applied to the impeller 20 in anon-contact manner (see FIG. 5). As shown in FIG. 5, the rotation center C3 of the drive motor 31 is arranged between the support shaft 7b and the inner peripheral surface 7a.

The drive motor 31 includes a control unit (not shown) that controls the rotation speed and rotation direction of the rotating magnet. The control unit can set the rotation speed and rotation direction of the rotating magnet to arbitrary values by adjusting the supply voltage to the drive motor 31 and the like. That is, the drive motor 31 can control the rotation speed and rotation direction of the impeller 20 by the control unit. The arrangement and number of the drive motors 31 can be arbitrarily determined as long as they can apply a magnetic field to the impeller 20 of the liquid flow structure 1.

As described above, in the liquid delivery device 100 having such a configuration, the support portion 9 configured by the outer periphery 7c of the support shaft 7b and the inner circle 21a of the annular portion 21 has a gap 24 for the annular portion 21 to be rotatable (FIG. 5). In the liquid delivery device 100, since the rotation center C3 of the drive motor 31 is arranged between the support shaft 7b and the inner peripheral surface 7a, the impeller 20 is rotated by the magnetic force of the drive motor 31 while being attracted to the rotation center C3 of the drive motor 31. That is, in the liquid delivery device 100, the drive motor 31 is arranged so that the impeller 20 can rotate with the gap 24 being biased in the contact range between the outer periphery 7c of the support shaft 7b and the inner circle 21a of the annular portion 21. Thus, the liquid delivery device 100 can rotate the impeller 20 in a state where the gap 24 is biased in the contact range between the outer periphery 7c of the support shaft 7b and the inner circle 21a of the annular portion 21, that is, in a state where the rotation center C2 of the impeller 20 converges in one direction. Since the outer periphery 7c of the support shaft 7b and the inner circle 21a of the annular portion 21 are often concentrated and contacted at a specific point, this is referred to as a point contact method in this specification. Therefore, the liquid delivery device 100 can converge the rotation position of the impeller 20 to a specific range narrower than that of the conventional art. Thereby, the liquid delivery device 100 can rotate the impeller 20 more stably.

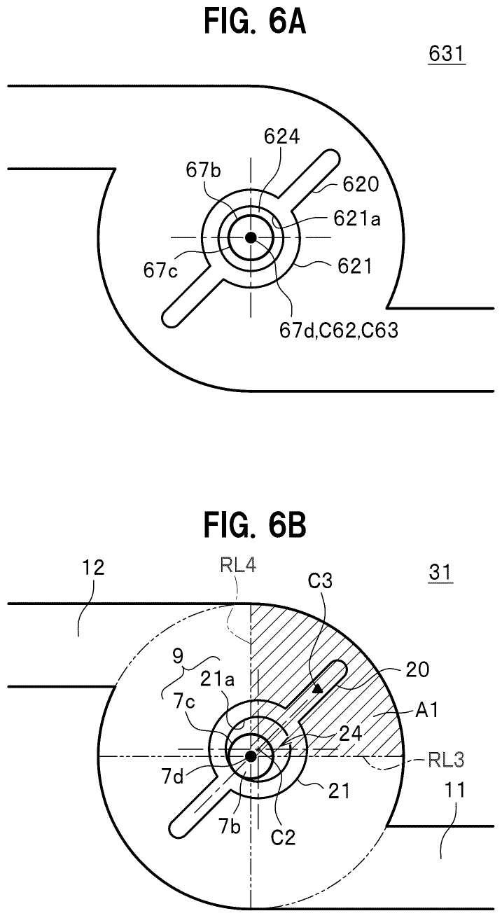

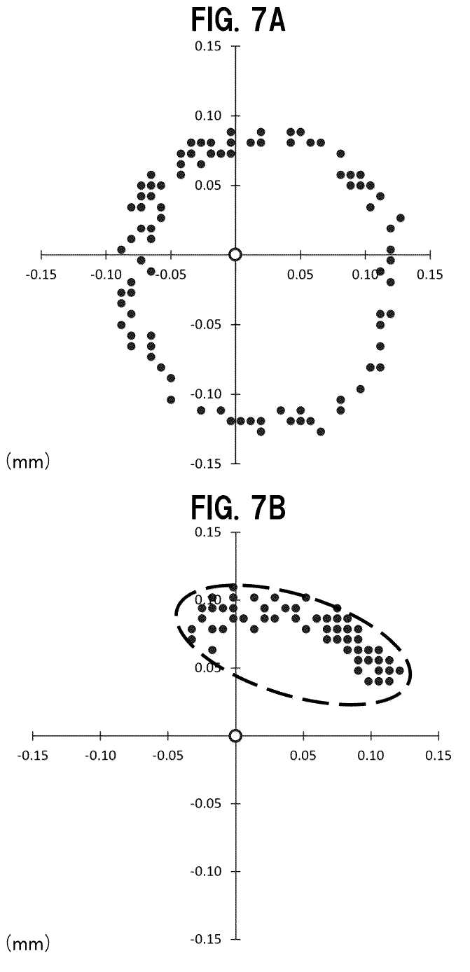

Here, FIGS. 6 and 7 are explanatory diagrams showing a difference in the rotation center position of the impeller between the conventional method and the present embodiment (point contact method) with respect to the first embodiment.

FIG. 6A is an explanatory diagram showing the relationship between the central axis 67d of the support shaft 67b and the rotation center C62 of the impeller 620 in the conventional method. FIG. 6B is an explanatory diagram showing the relationship between the center point 7d of the support shaft 7b and the rotation center C2 of the impeller 20 in the point contact method. In FIG. 6A, “●” represents the central axis 67d of the support shaft 67b. The cross of dash-dot lines and the intersection thereof represent the rotation center C62 of the impeller 620. In FIG. 6B, “●” represents the center point 7d of the support shaft 7b. Further, in the same figure, “▴” represents the rotation center C3 of the drive motor 31. The cross of dash-dot lines and the intersection thereof represent the rotation center C2 of the impeller 20.

FIG. 7A is an explanatory diagram showing the degree of variation in the rotation center C62 of the impeller 620 in the conventional method. FIG. 7B is an explanatory diagram showing the degree of variation in the rotation center C2 of the impeller 20 in the point contact method.

In the conventional method shown in FIG. 6A, the central axis 67d of the support shaft 67b and the rotation center C63 of the drive motor 631 are aligned, whereby the central axis 67d of the support shaft 67b and the rotation center C62 of the impeller 620 are aligned to rotate the impeller 620. Since these centers are aligned, this is referred to as the centering method in this specification.

As shown in FIG. 6A, in the centering method, which is the conventional method, there is a gap 624 between the outer periphery 67c of the support shaft 67b and the inner circle 621a of the annular portion 621 of the impeller 620. In the impeller 620 in the centering method, since the position of the rotation center C62 within the range of the gap 624 is not particularly limited, the position of the rotation center C62 can freely move within the range of the gap 624.

On the other hand, in the point contact method in the present embodiment shown in FIG. 6B, the rotation center C3 of the drive motor 31 is separated from the center point 7d of the support shaft 7b, and the impeller 20 is rotated by the magnetic force of the drive motor 31 while being attracted in a specific direction (the direction of the rotation center C3). Thereby, in the point contact method in this embodiment, the impeller 20 can be rotated without aligning the center point 7d of the support shaft 7b and the rotation center C2 of the impeller 20.

As shown in FIG. 6B, also in the point contact method in this embodiment, there is a gap 24 between the outer periphery 7c of the support shaft 7b and the inner circle 21a of the annular portion 21 of the impeller 20. However, in the point contact method in the present embodiment, the drive motor 31 is arranged so that the impeller 20 can rotate with the gap 24 being biased in the contact range between the outer periphery 7c of the support shaft 7b and the inner circle 21a of the annular portion 21. Thereby, in the point contact method in this embodiment, the movable range of the rotation center C2 of the impeller 20 within the range of the gap 24 is limited.

In FIG. 7A, “∘” at the intersection of the horizontal axis and the vertical axis indicates the position of the central axis 67d of the support shaft 67b, and “●” indicates the position of the rotation center C62 of the impeller 620 analyzed from images obtained by continuous shooting, which is plotted. The numerical values on the horizontal axis and the vertical axis in FIG. 7A indicate the distances from the intersection “∘”, and the unit thereof is millimeter (mm).

As shown in FIG. 7A, in the case of the centering method, which is the conventional method, it can be seen that the rotation center C62 of the impeller 620 is generally scattered around the central axis 67d of the support shaft 67b. That is, it can be seen that in the centering method, the impeller 620 is rotated (self-rotated) while the rotation center C62 thereof roughly revolves around the central axis 67d of the support shaft 67b.

In FIG. 7B, “●” at the intersection of the horizontal axis and the vertical axis indicates the position of the center point 7d of the support shaft 7b, and “●” indicates the position of the rotation center C2 of the impeller 20 analyzed from images obtained by continuous shooting, which is plotted. The numerical values on the horizontal axis and the vertical axis in FIG. 7B indicate the distances from the intersection “●”, and the unit thereof is millimeter (mm).

As shown in FIG. 7B, in the case of the point contact method in this embodiment, it can be seen that the rotation center C2 of the impeller 20 converges to some extent in a specific range (the upper right range in FIG. 7B) with respect to the center point 7d of the support shaft 7b.

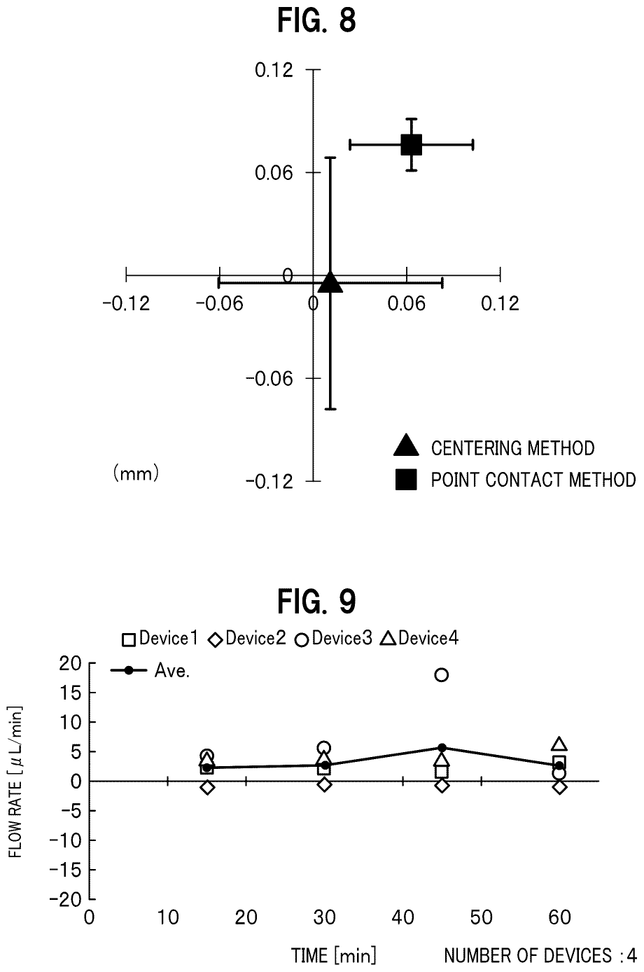

FIG. 8 is also an explanatory diagram showing a difference in the rotation center position of the impeller 20 between the conventional method and the present embodiment (point contact method) with respect to the first embodiment. The numerical values on the horizontal axis and the vertical axis in FIG. 8 indicate the distances from the center point (0, 0), and the unit thereof is millimeter (mm). FIG. 8 shows the results of investigating the positions of the rotation center C62 of the impeller 620 and the rotation center C2 of the impeller 20 by using a support shaft 7b having a diameter of about 0.7 mm and an impeller 20 (with a maximum length of about 3 mm) consisting of an annular portion 21 having an inner circle 21a with a diameter of about 0.8 mm and two blade portions 22, 22 (each about 1 mm in length) extending radially outward from the outer peripheral surface of the annular portion 21, and by rotating the impeller 20 with the drive motor 31.

Here, in the centering method, the center point 67d of the support shaft 67b and the rotation center C63 of the drive motor 631 are aligned (FIG. 6A).

On the other hand, in the point contact method, the rotation center C3 of the drive motor 31 is arranged away from the center point 7d of the support shaft 7b (FIG. 6B). Specifically, in the point contact method, in top-down view of the liquid delivery chamber 7, the rotation center C3 of the drive motor 31 is shifted by about 1 mm toward a position substantially midway between the first flow path 11 and the second flow path 12 on the side wall (inner peripheral surface 7a) of the liquid delivery chamber 7. That is, in FIG. 6B, the rotation center C3 of the drive motor 31 is arranged about 1 mm to the upper right from the center point 7d of the support shaft 7b.

In FIG. 8, “▴” indicates the position of the center point 67d of the support shaft 67b in the centering method, and the vertical and horizontal crosses indicate the range in which the rotation center C62 of the impeller 620 has moved. “▪” indicates the position of the center point 7d of the support shaft 7b in the point contact method, and the vertical and horizontal crosses indicate the vertical and horizontal ranges in which the rotation center C2 of the impeller 20 has moved.

As shown in FIG. 8, in the centering method, the rotation center C62 moved in the range of about −0.07 to 0.07 mm both vertically and horizontally. Further, as shown in FIG. 8, in the point contact method, the rotation center C2 moved in the range of about −0.015 mm to 0.015 mm vertically and about −0.03 mm to 0.03 mm horizontally. FIG. 8 also shows that the rotation center C2 of the impeller 20 converges in a certain range in the point contact method in the present embodiment more effectively than in the centering method. From this, it can be seen that the impeller 20 can be more stably rotated (at a stable position) in the point contact method than in the centering method.

FIG. 9 is a graph showing the results of measuring the average flow rate of the impeller 20 in the point contact method over time, which was measured when the investigation regarding FIG. 8 was conducted. The horizontal axis represents time [min], and the vertical axis represents the average flow rate [μL/min]. Device 1 to 4 indicate the device numbers of the liquid delivery devices 100 used. That is, the measurement is performed using four liquid delivery devices 100 manufactured with the same design. The line graph indicated by “●” indicates the average (Ave.) of the average flow rates of Device 1 to 4 at each measurement time.

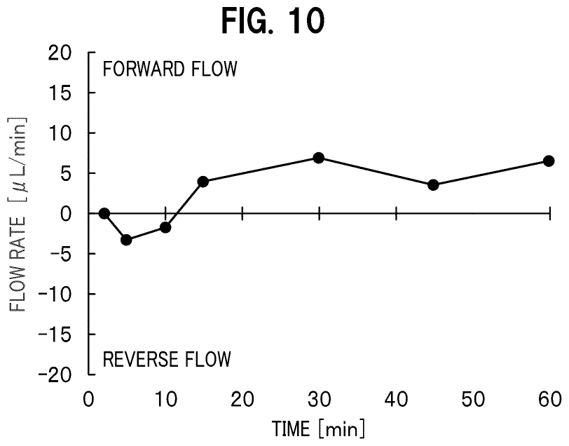

Although the four devices have the same design drawing, since they are self-made by the present inventors, there are slight design errors in each device. Therefore, although there were variations among the devices, as shown in FIG. 9—unlike the situation shown in FIG. 10, which will be described later—when employing the point contact method, there were almost no instances in which the average flow rate went negative over time and then returned to a value close to the original; in other words, the liquid hardly flowed backward, both among the four devices themselves and in the “●” line graph, which represents the average of the average flow rates of the four devices at each measurement time. Therefore, the point contact method can make the direction of liquid flow constant.

From the above description, as shown in FIG. 6B, when the liquid delivery chamber 7 of the liquid delivery device 100 has a point-symmetrical positional relationship between the first flow path 11 and the second flow path 12 with respect to the support shaft 7b in top-down view, the rotation center C2 of the impeller 20 is located in the area A1 corresponding to the first quadrant with respect to the third reference line RL3 passing through the center point 7d and parallel to the inflow direction of the liquid to the liquid delivery chamber 7 and the fourth reference line RL4 passing through the center point 7d and orthogonal to the third reference line RL3 in the concentric circle centered on the center point 7d of the support shaft 7b, and the drive motor 31 is arranged so that the impeller 20 can rotate with the gap 24 being biased (that is, the rotation center C3 of the drive motor 31 is arranged in the area A1), it can be seen that the liquid delivery can be continuously performed with the direction of liquid flow being constant and the flow rate being stable. The third reference line RL3 described above can be considered as, for example, the x-axis, and the fourth reference line RL4 described above can be considered as, for example, the y-axis.

### (Preferred Aspects)

When the liquid delivery chamber 7 of the liquid delivery device 100 has a point-symmetrical positional relationship between the first flow path 11 and the second flow path 12 with respect to the support shaft 7b in top-down view, since the first flow path 11 and the second flow path 12 have the same shape, it is easy to flow liquid into both of them, and it is also easy to flow liquid out of both of them. Although it is not clear whether this is the cause, in the centering method, which is the conventional method, even when the impeller 20 is continuously rotated in a certain direction (specifically, the counterclockwise forward flow F1 direction), although infrequently, a backflow phenomenon in which the liquid flows backward for a short time may occur. FIG. 10 is a graph showing a backflow phenomenon that occurred when a liquid delivery chamber 7 in which the first flow path 11 and the second flow path 12 are provided point-symmetrically with respect to the support shaft 7b is used in the liquid delivery device 100. In the figure, the horizontal axis represents time [min], and the vertical axis represents flow velocity [μL/min]. The vertical axis is centered on 0 [μL/min], with the +side above indicating the forward flow F1 and the—side below indicating the reverse flow F2.

As shown in FIG. 10, in the centering method, which is the conventional method, in the case of the liquid delivery chamber 7 in which the first flow path 11 and the second flow path 12 are provided in a point-symmetrical positional relationship with respect to the support shaft 7b, the liquid flows in the reverse flow F2 direction for a short time immediately after the start of operation, and then the liquid flows stably in the forward flow F1 direction for a long time (there were also devices in which the backflow phenomenon did not occur), but by using the point contact method, as shown in FIG. 9, the direction of liquid flow was generally constant and the flow rate was stable. Thus, when the liquid delivery chamber 7 (point symmetry) is used, good results were obtained in long-time operation by using the point contact method, but it was considered that more stable liquid delivery could be performed by further devising. As a result of various studies, it was found that more stable liquid delivery can be more reliably achieved by using a liquid delivery chamber 7 in which the first flow path 11 and the second flow path 12 are in an asymmetrical or line-symmetrical positional relationship with respect to the support shaft 7b in top-down view.

Hereinafter, preferred specific aspects of the liquid delivery device 100 will be described.

### Second Embodiment

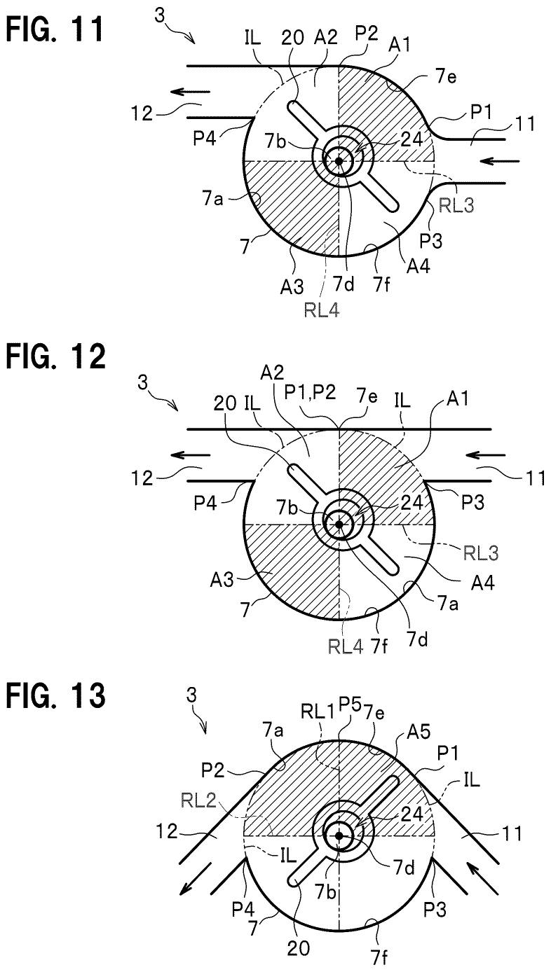

FIG. 11 is an explanatory diagram of the liquid delivery section 3 of the liquid delivery device 100 according to the second embodiment.

As shown in FIG. 11, the liquid delivery chamber 7 of the liquid delivery section 3 in the second embodiment has a circular shape in top-down view. The circular liquid delivery chamber 7 is shown by a virtual line IL.

Further, in the liquid delivery chamber 7 of this aspect, in top-down view, a length of a first wall portion 7e, which is formed by a first connection portion P1 connected to the first flow path 11, a second connection portion P2 connected to the second flow path 12, and a circular inner peripheral surface 7a between the first connection portion PT and the second connection portion P2 is shorter than a length of a second wall portion 7f, which is formed by a third connection portion P3 connected to the first flow path 11, a fourth connection portion P4 connected to the second flow path 12, and a circular inner peripheral surface 7a between the third connection portion P3 and the fourth connection portion P4.

Further, in this aspect, the formation direction of the first flow path 11 with respect to the liquid delivery chamber 7 and the formation direction of the second flow path 12 with respect to the liquid delivery chamber 7 are parallel, but the first flow path 11 and the second flow path 12 are formed to be offset with respect to each other via the liquid delivery chamber 7.

That is, in the second embodiment, as shown in FIG. 11, the first flow path 11 and the second flow path 12 are provided at positions that are asymmetrical with respect to the support shaft 7b of the liquid delivery chamber 7.

In this aspect, the impeller 20 rotates in the same direction as the outflow direction of the liquid flowing along the first wall portion 7e. That is, in this aspect, the impeller 20 rotates in the direction from the first flow path 11 to the second flow path 12 along the first wall portion 7e.

Further, in this aspect, in the concentric circle centered on the center point 7d of the support shaft 7b, the liquid delivery chamber 7 includes the first flow path 11 at a position including the boundary line between the first quadrant and the fourth quadrant with respect to the third reference line RL3 passing through the center point 7d and parallel to the inflow direction of the liquid to the liquid delivery chamber 7 and the fourth reference line RL4 passing through the center point 7d and orthogonal to the third reference line RL3, and includes the second flow path 12 at a position corresponding to the second quadrant with respect to the third reference line RL3 and the fourth reference line RL4. The third reference line RL3 described above can be considered as, for example, the x-axis, and the fourth reference line RL4 described above can be considered as, for example, the y-axis.

In this aspect, as shown in FIG. 11, the drive motor 31 is arranged so that the rotation center C2 of the impeller 20 (see FIGS. 5 and 6B) is located in the area A1 corresponding to the first quadrant or the area A3 corresponding to the third quadrant with respect to the third reference line RL3 and the fourth reference line RL4, and the impeller 20 can rotate with the gap 24 being biased.

In the second embodiment, since the drive motor 31 is arranged so that the rotation center C2 of the impeller 20 is located in the area A1 corresponding to the first quadrant or the area A3 corresponding to the third quadrant, and the impeller 20 can rotate with the gap 24 being biased, the rotation (rotation position) of the impeller 20 is stabilized. Therefore, the liquid delivery device 100 according to the second embodiment can continuously deliver liquid in a constant flow direction with a stable flow rate.

### Third Embodiment

FIG. 12 is an explanatory diagram of the liquid delivery section 3 of the liquid delivery device 100 according to the third embodiment.

As shown in FIG. 12, the liquid delivery chamber 7 of the liquid delivery section 3 in the third embodiment also has a circular shape in top-down view.

Further, in the liquid delivery chamber 7 of this aspect, in top-down view, a length of a first wall portion 7e, which is formed by a first connection portion P1 connected to the first flow path 11, a second connection portion P2 connected to the second flow path 12, and a circular inner peripheral surface 7a between the first connection portion P1 and the second connection portion P2 is shorter than a length of a second wall portion 7f, which is formed by a third connection portion P3 connected to the first flow path 11, a fourth connection portion P4 connected to the second flow path 12, and a circular inner peripheral surface 7a between the third connection portion P3 and the fourth connection portion P4. In this aspect, as shown in FIG. 12, the first connection portion P1 and the second connection portion P2 may be in direct contact with each other. In this case, the length of the first wall portion 7e formed by the circular inner peripheral surface 7a between the first connection portion P1 and the second connection portion P2 is 0.

That is, as shown in FIG. 12, in this aspect, the first flow path 11 and the second flow path 12 are formed on the same straight line via the liquid delivery chamber 7.

Therefore, in the third embodiment, as shown in FIG. 12, the first flow path 11 and the second flow path 12 are provided at positions that are line-symmetrical with respect to the support shaft 7b of the liquid delivery chamber 7 (with respect to the fourth reference line RL4, which will be described later).

In this aspect, as an example, the impeller 20 can be rotated in the same direction as the outflow direction of the liquid flowing along the first wall portion 7e. That is, in this aspect, as an example, the impeller 20 can be rotated in the direction from the first flow path 11 to the second flow path 12 along the first wall portion 7e.

Further, in this aspect, as shown in FIG. 12, in the concentric circle centered on the center point 7d of the support shaft 7b, the liquid delivery chamber 7 includes the first flow path 11 at a position corresponding to the first quadrant with respect to the third reference line RL3 passing through the center point 7d and parallel to the inflow direction of the liquid to the liquid delivery chamber 7 and the fourth reference line RL4 passing through the center point 7d and orthogonal to the third reference line RL3, and includes the second flow path 12 at a position corresponding to the second quadrant with respect to the third reference line RL3 and the fourth reference line RL4. The third reference line RL3 described above can be considered as, for example, the x-axis, and the fourth reference line RL4 described above can be considered as, for example, the y-axis.

In this aspect, as shown in FIG. 12, the drive motor 31 is arranged so that the rotation center C2 of the impeller 20 (see FIGS. 5 and 6B) is located in the area A1 corresponding to the first quadrant or the area A3 corresponding to the third quadrant with respect to the third reference line RL3 and the fourth reference line RL4, and the impeller 20 can rotate with the gap 24 being biased.

In the third embodiment, since the drive motor 31 is arranged so that the rotation center C2 of the impeller 20 is located in the area A1 corresponding to the first quadrant or the area A3 corresponding to the third quadrant, and the impeller 20 can rotate with the gap 24 being biased, the rotation (rotation position) of the impeller 20 is stabilized. Therefore, the liquid delivery device 100 according to the third embodiment can continuously deliver liquid in a constant flow direction with a stable flow rate.

### Fourth Embodiment

FIG. 13 is an explanatory diagram of the liquid delivery section 3 of the liquid delivery device 100 according to the fourth embodiment.

As shown in FIG. 13, the liquid delivery chamber 7 of the liquid delivery section 3 in the fourth embodiment also has a circular shape in top-down view.

Further, in the liquid delivery chamber 7 of this aspect, in top-down view, a length of a first wall portion 7e, which is formed by a first connection portion P1 connected to the first flow path 11, a second connection portion P2 connected to the second flow path 12, and a circular inner peripheral surface 7a between the first connection portion P1 and the second connection portion P2 is shorter than a length of a second wall portion 7f, which is formed by a third connection portion P3 connected to the first flow path 11, a fourth connection portion P4 connected to the second flow path 12, and a circular inner peripheral surface 7a between the third connection portion P3 and the fourth connection portion P4.

Further, in this aspect, the first flow path 11 and the second flow path 12 are provided so that the inflow direction of the liquid flowing into the liquid delivery chamber 7 and the outflow direction of the liquid flowing out of the liquid delivery chamber 7 are aligned continuously by the arc of the first wall portion 7e. The angle formed by the first flow path 11 and the second flow path 12 can be, for example, 90°.

That is, in the fourth embodiment, as shown in FIG. 13, the first flow path 11 and the second flow path 12 are provided at positions that are line-symmetrical with respect to the support shaft 7b of the liquid delivery chamber 7 (with respect to the first reference line RL1, which will be described later).

In this aspect, as an example, the impeller 20 can be rotated in the same direction as the outflow direction of the liquid flowing along the first wall portion 7e. That is, in this aspect, as an example, the impeller 20 can be rotated in the direction from the first flow path 11 to the second flow path 12 along the first wall portion 7e.

In this aspect, as shown in FIG. 13, the drive motor 31 is arranged so that the area is bisected by the second reference line RL2 passing through the center point 7d of the support shaft 7b and orthogonal to the first reference line RL1 passing through the center point 7d and the point P5 bisecting the length of the first wall portion 7e, and the rotation center C2 of the impeller 20 (see FIGS. 5 and 6B) is located in the semicircular area A5 including the first wall portion 7e, and the impeller 20 can rotate with the gap 24 being biased. The second reference line RL2 described above can be considered as, for example, the x-axis, and the first reference line RL1 described above can be considered as, for example, the y-axis.

In the fourth embodiment, since the drive motor 31 is arranged so that the rotation center C2 of the impeller 20 is located in the semicircular area A5 and the impeller 20 can rotate with the gap 24 being biased, the rotation (rotation position) of the impeller 20 is stabilized. Therefore, the liquid delivery device 100 according to the fourth embodiment can continuously deliver liquid in a constant flow direction with a stable flow rate.

### Fifth Embodiment

In this embodiment, in any case where the liquid delivery chamber 7 of the liquid delivery device 100 has a point-symmetrical positional relationship (first embodiment) or a line-symmetrical positional relationship (third embodiment, fourth embodiment) between the first flow path 11 and the second flow path 12 with respect to the support shaft 7b in top-down view, since the first flow path 11 and the second flow path 12 are provided in a highly symmetrical manner, the liquid delivery direction can be easily adjusted by changing the rotation direction of the impeller 20. For example, in a certain cell culture, the impeller 20 can be rotated in the rotation direction D1 (counterclockwise direction) to deliver the liquid in the forward flow F1 direction, and in another cell culture, the impeller 20 can be rotated in the rotation direction D2 (clockwise direction) to deliver the liquid in the reverse flow F2 direction. In this embodiment, these can be arbitrarily used properly.

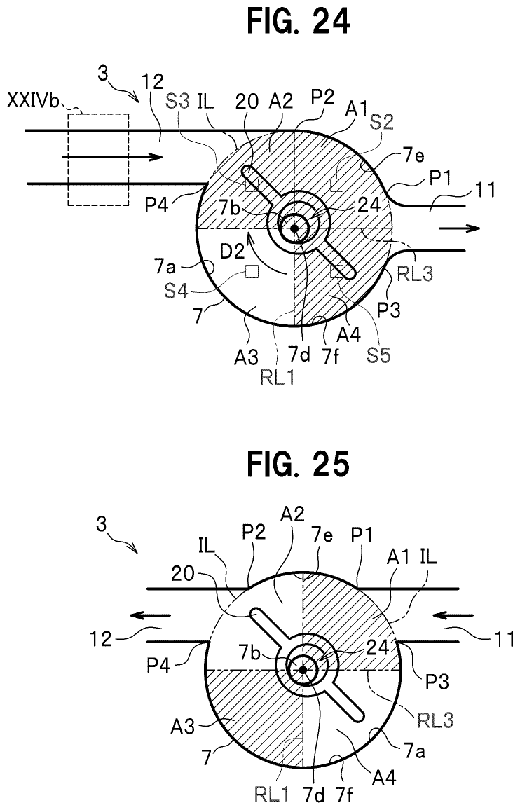

Further, in this embodiment, also when the liquid delivery chamber 7 of the liquid delivery device 100 has an asymmetrical positional relationship between the first flow path 11 and the second flow path 12 with respect to the support shaft 7b in top-down view (second embodiment), the liquid delivery direction can be adjusted by changing the rotation direction of the impeller 20. However, in this aspect, since the first flow path 11 and the second flow path 12 are not provided symmetrically, the arrangement position of the drive motor 31 capable of delivering the liquid in a constant flow direction with a stable flow rate is different from the area described in the second embodiment. This point will be described later in Example 5 (see FIG. 24).

### [Liquid Delivery Method]

Next, a liquid delivery method of the liquid delivery device 100 described above will be described.

The device body 10 is placed on the drive device 30 in accordance with the positioning portions 32 of the drive device 30. Thereby, in the liquid delivery device 100, the drive motor 31 is arranged so that the impeller 20 can rotate with the gap 24 being biased in the contact range between the outer periphery 7c of the support shaft 7b and the inner circle 21a of the annular portion 21.

Next, the drive motor 31 of the drive device 30 is operated. As an example, as shown in FIG. 5, the liquid delivery device 100 applies a rotational driving force in the rotation direction D1 (counterclockwise direction) around the rotation center C2 to the impeller 20 by the drive motor 31. Thereby, the impeller 20 rotates in the rotation direction D1 (counterclockwise direction) around the rotation center C2. That is, the impeller 20 rotates in the same direction as the outflow direction of the liquid flowing along the first wall portion 7e. Here, the impeller 20 rotates in the direction from the first flow path 11 to the second flow path 12 (counterclockwise direction) along the first wall portion 7e.

Due to the rotation of the impeller 20, a rotational flow of the liquid in the rotation direction D1 (counterclockwise direction) around the rotation center C2 of the impeller 20 is generated in the liquid delivery chamber 7. According to the rotational flow in the liquid delivery chamber 7, the liquid flows into the liquid delivery chamber 7 from the first flow path 11 through the liquid inlet/outlet 14 and flows to the second flow path 12 through the liquid inlet/outlet 15 (the liquid flows in the forward flow F1 direction). In this example, the liquid inlet/outlet 14 serves as an inlet. The liquid inlet/outlet 15 serves as an outlet.

The reason why the flow from the first flow path 11 to the second flow path 12 via the liquid delivery chamber 7 is generated can be estimated as follows. Due to the rotation of the impeller 20, vortices having different sizes are generated at positions close to the liquid inlet/outlet 14 and the liquid inlet/outlet 15, respectively. The flow direction (rotation direction) of the vortex is, for example, the same as the rotation direction D1 of the impeller 20. Due to the generation of the vortices having different shapes, sizes, and flow velocities, the liquid pressure at the liquid inlet/outlet 15 becomes lower than the liquid pressure at the liquid inlet/outlet 14. As a result, the liquid in the liquid delivery chamber 7 flows from the liquid inlet/outlet 14 to the liquid inlet/outlet 15. Therefore, it is considered that the liquid is delivered from the first flow path 11 to the second flow path 12 via the liquid delivery chamber 7.

As shown in FIG. 4, due to the generation of the liquid flow from the first flow path 11 to the second flow path 12 via the liquid delivery chamber 7 (the liquid flow in the direction of the arrow in FIG. 4), the liquid in the first reservoir 5 flows into the first flow path 11. The liquid in the second flow path 12 flows into the second reservoir 6.

The flow rate of the liquid from the first flow path 11 to the second flow path 12 via the liquid delivery chamber 7 can be adjusted by the rotation speed of the impeller 20. To set the rotation speed of the impeller 20, the number of rotations of the rotating magnet (not shown) of the drive motor 31 is adjusted. Thereby, the liquid delivery device 100 can circulate the liquid in the loop-shaped flow path 4 at an arbitrary flow rate.

Further, in this embodiment, the liquid delivery direction can be adjusted by the rotation direction of the impeller 20. That is, by reversing the rotation direction of the drive motor 31 and rotating the impeller 20 in the rotation direction D2 (clockwise direction), the liquid delivery direction can be set to the reverse flow F2 direction.

Since the liquid delivery device 100 delivers liquid by the impeller 20 in the liquid delivery chamber 7, the number of operations other than setting the rotation and stop of the impeller 20 can be reduced. Therefore, the operation for liquid delivery is easy. Therefore, convenience can be improved.

Since the structure of the liquid delivery device 100 for delivering liquid by the impeller 20 in the liquid delivery chamber 7 is simple, it is easy to reduce the size thereof. Further, since the liquid delivery structure is simple, cost reduction can be achieved.

### Example 1

### First Example

### (Study 1 on the Shape of the Liquid Delivery Section 3 of the Device Body 10)

Next, the liquid delivery device according to the present invention will be described with reference to examples.

A device body 10 made of PMMA and having a rectangular shape in top-down view, which is formed in a block shape, was prepared. In [First Example], the PMMA device body 10 was cut with a three-dimensional plotter to form a liquid flow structure 1 (a liquid flow section 2, a liquid delivery section 3, and a loop-shaped flow path 4) as shown in FIG. 4.

When forming the liquid flow section 2 and the liquid delivery section 3 (liquid delivery chamber 7), the shapes thereof were changed to prepare four device bodies 10 according to Examples 1 to 3 and three device bodies 10 according to Example 4. The liquid flow structure 1 (liquid flow section 2, liquid delivery section 3, and loop-shaped flow path 4) in each device body 10 was formed using a three-dimensional plotter.

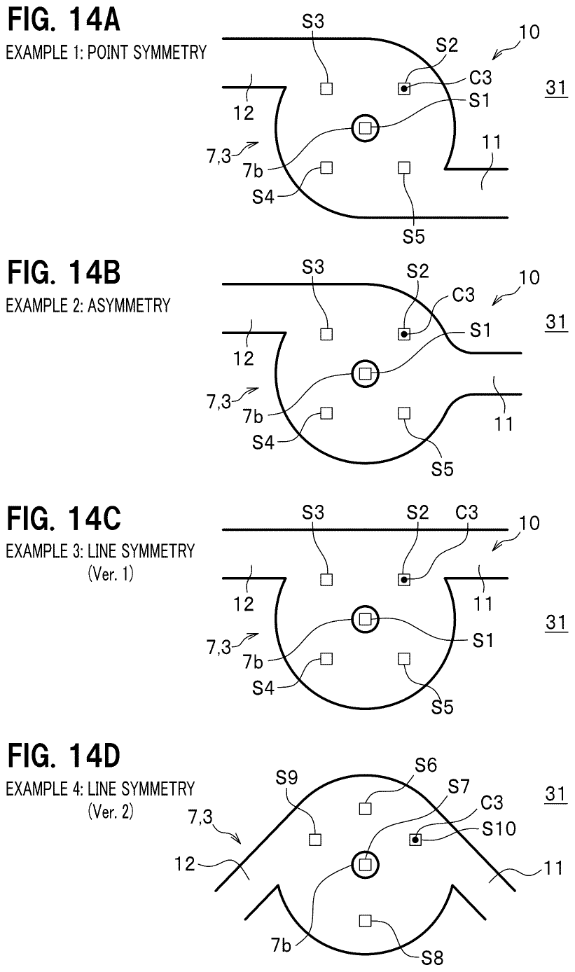

Here, FIGS. 14A to 14D are explanatory diagrams showing the shape of the liquid delivery section 3 of the device body 10 and the arrangement position of the rotation center C3 of the drive motor 31, which will be described later, according to Examples 1 to 4, respectively.

As shown in FIG. 14A, in the liquid delivery section 3 of the device body 10 according to Example 1, the first flow path 11 and the second flow path 12 are point-symmetrical with respect to the support shaft 7b of the liquid delivery chamber 7 (see FIG. 6B).

As shown in FIG. 14B, in the liquid delivery section 3 of the device body 10 according to Example 2, the first flow path 11 and the second flow path 12 are asymmetrical with respect to the support shaft 7b of the liquid delivery chamber 7 (see FIG. 11).

As shown in FIG. 14C, in the liquid delivery section 3 of the device body 10 according to Example 3, the first flow path 11 and the second flow path 12 are line-symmetrical (line symmetry Ver. 1) with respect to the support shaft 7b of the liquid delivery chamber 7 (see FIG. 12).

As shown in FIG. 14D, in the liquid delivery section 3 of the device body 10 according to Example 4, the first flow path 11 and the second flow path 12 are line-symmetrical (line symmetry Ver. 2) with respect to the support shaft 7b of the liquid delivery chamber 7 (see FIG. 13).

The behavior of the impeller 20 and the flow rate (average flow rate) of the solution when the impeller 20 is rotated by arranging the rotation center C3 of the drive motor 31 at each of the five positions: center S1, upper right S2, upper left S3, lower left S4, and lower right S5 shown by “n” in each of FIGS. 14A to 14C, and top S6, center S7, bottom S8, upper left S9, and upper right S10 shown by “□” in FIG. 14D were investigated. FIGS. 14A to 14C show that the rotation center C3 of the drive motor 31 is arranged at the upper right S2. FIG. 14D shows that the rotation center C3 of the drive motor 31 is arranged at the upper right S10.

The diameter of the support shaft 7b of the liquid delivery chamber 7 was about 0.7 mm.

An impeller 20 made of a magnetic material was used. The impeller 20 has an inner circle 21 a of the annular portion 21 having a diameter of about 0.8 mm and two blade portions 22, 22 (each having a length of about 1 mm) extending radially outward from the outer peripheral surface of the annular portion 21 on the same straight line.

The annular portion 21 was rotatably supported by the support shaft 7b so that the impeller 20 could rotate around the support shaft 7b of the liquid delivery chamber 7.

The average flow path depth of the liquid delivery chamber 7, the first flow path 11, the second flow path 12, and the return flow path 13 in Examples 1 to 4 was 350 μm.

Then, the first reservoir 5 and the second reservoir 6 were filled with a solution for flow rate measurement, and the liquid flow structure 1 was filled with the solution.

In this state, the drive motor 31 was driven to rotate the impeller 20 in the rotation direction D1 (counterclockwise direction), and the flow rate was measured.

The solution for flow rate measurement, the driving time of the drive motor 31, and the rotation speed of the impeller 20 were as follows.
Solution: Fluorescent microbead dispersion solution with a diameter of about 1.0 μm
Driving time: 60 min (start of driving the drive motor 31=0 min)
Rotation speed: about 2400 rpm
Rotation direction: rotation direction D1 (counterclockwise direction) in top-down view

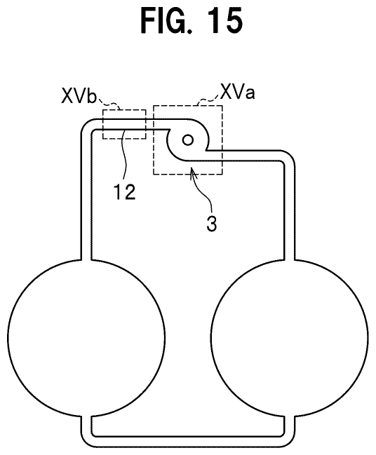

FIG. 15 is an explanatory diagram showing how the impeller 20 rotates and how the average flow rate is measured in the examples.

A close-up camera was installed close to the XVa portion in FIG. 15, that is, the liquid delivery section 3, and the state of rotation of the impeller 20 was continuously photographed. Then, the rotation center C2 of the impeller 20 was plotted for each continuous image using the image processing software ImageJ, and the behavior of the impeller 20 was measured. FIG. 7B shows the plotted rotation center C2 of the impeller 20 in the device body 10 according to Example 1 (point contact method). FIG. 7A shows the plotted rotation center C2 of the impeller 20 when the impeller 20 was rotated by reproducing the centering method, which is the conventional method, by aligning the center point 7d of the support shaft 7b and the rotation center C2 of the impeller 20 at the time of investigating the device body 10 according to Example 1. Therefore, FIG. 7A shows results corresponding to a comparative example.

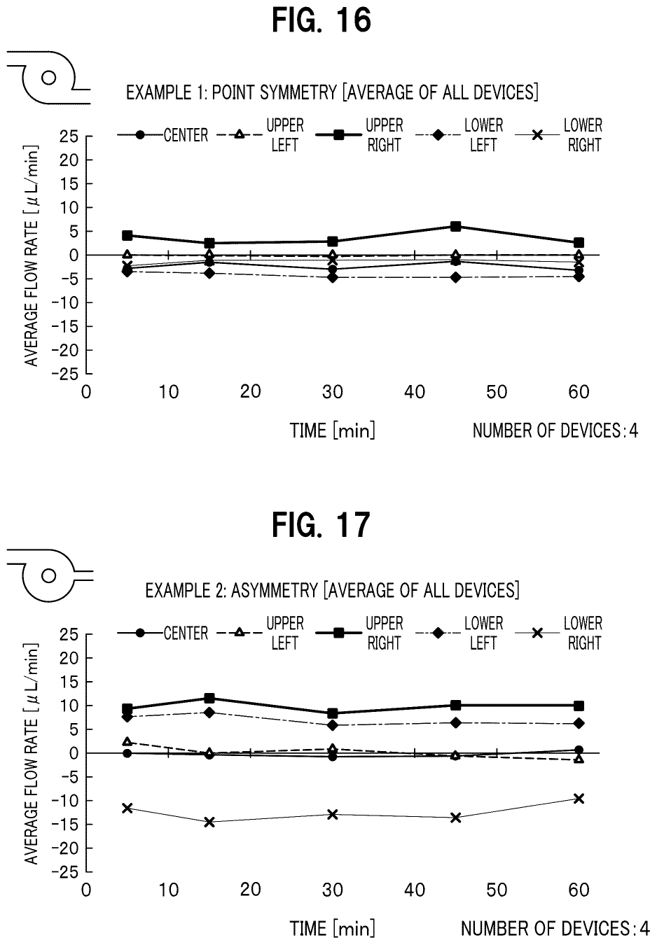

A close-up camera was installed close to the XVb portion in FIG. 15, that is, the second flow path 12, and the state of the solution flowing through the second flow path 12 was continuously photographed. Then, the average flow rate of each device (liquid delivery device 100) in Examples 1 to 4 was calculated using PIV analysis software (Flow Expert 2D2C). The results are shown in FIGS. 16 to 19.

FIG. 16 is a graph showing changes over time in the average flow rate when the rotation center C3 of the drive motor 31 is arranged at five locations: center S1, upper right S2, upper left S3, lower left S4, and lower right S5 (see FIG. 14A) of the liquid delivery chamber 7 (point symmetry) of the liquid delivery device 100 according to Example 1 in FIG. 14A (number of devices used: 4).

FIG. 17 is a graph showing changes over time in the average flow rate when the rotation center C3 of the drive motor 31 is arranged at five locations: center S1, upper right S2, upper left S3, lower left S4, and lower right S5 (see FIG. 14B) of the liquid delivery chamber 7 (asymmetry) of the liquid delivery device 100 according to Example 2 in FIG. 14B (number of devices used: 4).

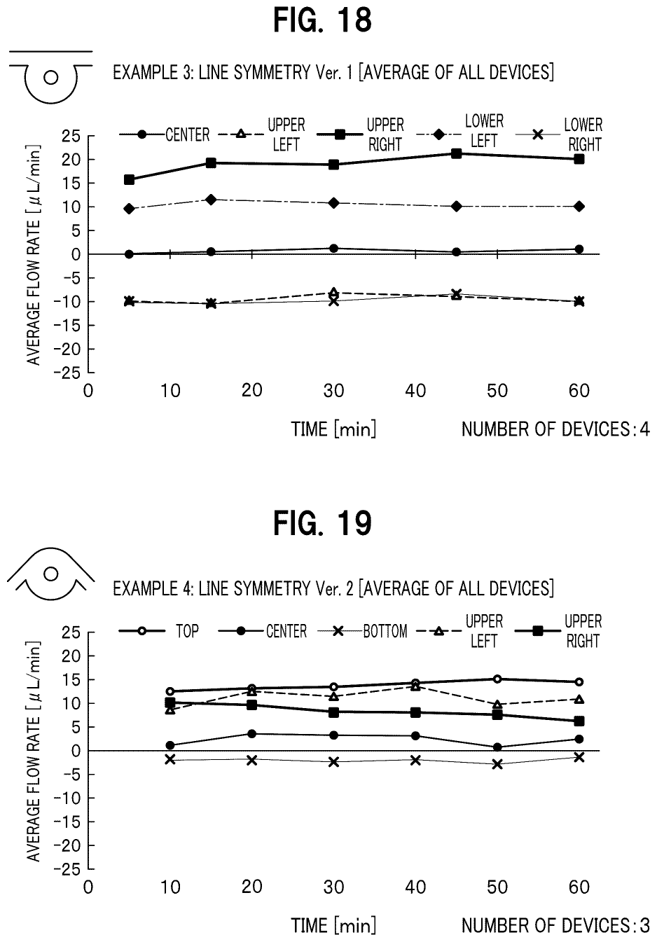

FIG. 18 is a graph showing changes over time in the average flow rate when the rotation center C3 of the drive motor 31 is arranged at five locations: center S1, upper right S2, upper left S3, lower left S4, and lower right S5 (see FIG. 14C) of the liquid delivery chamber 7 (line symmetry Ver. 1) of the liquid delivery device 100 according to Example 3 in FIG. 14C (number of devices used: 4).

FIG. 19 is a graph showing changes over time in the average flow rate when the rotation center C3 of the drive motor 31 is arranged at five locations: top S6, center S7, bottom S8, upper left S9, and upper right S10 (see FIG. 14D) of the liquid delivery chamber 7 (line symmetry Ver. 2) of the liquid delivery device 100 according to Example 4 in FIG. 14D (number of devices used: 3).

In FIGS. 16 to 19, the horizontal axis represents time [min], and the vertical axis represents the average flow rate [μL/min]. A positive value on the vertical axis indicates the average flow rate of the forward flow F1, and a negative value indicates the average flow rate of the reverse flow F2. Further, in FIGS. 16 to 19, the shape of the liquid delivery chamber 7 in Examples 1 to 4 is shown in the upper left. In Examples 1 to 3, continuous shooting was started 5 minutes after the start of driving the drive motor 31 (that is, the calculation of the flow rate was started), and continuous shooting was performed until 60 minutes after the start of driving the drive motor 31. In Example 4, continuous shooting was started 10 minutes after the start of driving the drive motor 31 (that is, the calculation of the flow rate was started), and continuous shooting was performed until 60 minutes after the start of driving the drive motor 31.

As shown in FIGS. 16 to 19, in all of the liquid delivery devices 100 according to Examples 1 to 4, the drive motor 31 is arranged so that the impeller 20 can rotate with the gap 24 being biased in the contact range between the outer periphery 7c of the support shaft 7b and the inner circle 21a of the annular portion 21. Therefore, all of these devices were able to continuously deliver liquid in a constant flow direction with a stable flow rate. Further, since the transition of the direction of liquid flow and the flow rate is stable in all of these devices, it is considered that the direction of liquid flow is similarly constant and the flow rate is also stable during the period from 0 to 5 minutes (Examples 1 to 3) or 10 minutes (Example 4) after the start of driving the drive motor 31. When the liquid delivery devices 100 according to Examples 1 to 4 are used for cell culture, the composition of the liquid medium circulating in the flow paths, the first reservoir 5, and the second reservoir 6 hardly changes in about 10 minutes from the start of culture, so that it is considered that cell culture can be performed more favorably than in the conventional art.

Specifically, as shown in FIG. 16 and FIG. 14A, in the liquid delivery device 100 according to Example 1, the first flow path 11 and the second flow path 12 are formed to be point-symmetrical with respect to the support shaft 7b.

The liquid delivery device 100 according to Example 1 was able to continuously deliver liquid in a constant flow direction with a stable flow rate at the upper right S2 position (see FIG. 6B) of the liquid delivery chamber 7 (point symmetry).

That is, when the liquid delivery chamber 7 of the liquid delivery device 100 has a point-symmetrical positional relationship between the first flow path 11 and the second flow path 12 with respect to the support shaft 7b in top-down view, the rotation center C2 of the impeller 20 is located in the area A1 corresponding to the first quadrant with respect to the third reference line RL3 passing through the center point 7d and parallel to the inflow direction of the liquid to the liquid delivery chamber 7 and the fourth reference line RL4 passing through the center point 7d and orthogonal to the third reference line RL3 in the concentric circle centered on the center point 7d of the support shaft 7b, and the drive motor 31 is arranged so that the impeller 20 can rotate in the rotation direction D1 (counterclockwise direction) in top-down view with the gap 24 being biased (that is, the rotation center C3 of the drive motor 31 is arranged in the area A1), the liquid delivery could be continuously performed with the direction of liquid flow being constant in the forward flow F1 direction and the flow rate being also constant.

Further, as shown in FIG. 16 and FIG. 14A, in the liquid delivery device 100 according to Example 1, the liquid delivery could be continuously performed with the direction of liquid flow being constant and the flow rate being also stable at the lower left S4 position of the liquid delivery chamber 7 (point symmetry).

That is, when the liquid delivery chamber 7 of the liquid delivery device 100 has a point-symmetrical positional relationship between the first flow path 11 and the second flow path 12 with respect to the support shaft 7b in top-down view, the rotation center C2 of the impeller 20 is located in the area A3 corresponding to the third quadrant with respect to the third reference line RL3 passing through the center point 7d and parallel to the inflow direction of the liquid to the liquid delivery chamber 7 and the fourth reference line RL4 passing through the center point 7d and orthogonal to the third reference line RL3 in the concentric circle centered on the center point 7d of the support shaft 7b, and the drive motor 31 is arranged so that the impeller 20 can rotate in the rotation direction D1 (counterclockwise direction) in top-down view with the gap 24 being biased (that is, the rotation center C3 of the drive motor 31 is arranged in the area A3), the liquid delivery could be continuously performed with the direction of liquid flow being constant in the reverse flow F2 direction and the flow rate being also constant.

As shown in FIG. 17 and FIG. 14B, in the liquid delivery device 100 according to Example 2, the formation direction of the first flow path 11 with respect to the liquid delivery chamber 7 and the formation direction of the second flow path 12 with respect to the liquid delivery chamber 7 are parallel, but the first flow path 11 and the second flow path 12 are formed to be offset with respect to each other via the liquid delivery chamber 7.

The liquid delivery device 100 according to Example 2 was able to continuously deliver liquid with a constant flow direction and a stable flow rate at the upper right S2 and lower left S4 positions of the liquid delivery chamber 7 (asymmetry).

That is, in the liquid delivery device 100, the liquid delivery chamber 7 includes the first flow path 11 at a position including the boundary line between the first quadrant and the fourth quadrant with respect to the third reference line RL3 passing through the center point 7d and parallel to the inflow direction of the liquid to the liquid delivery chamber 7 and the fourth reference line RL4 passing through the center point 7d and orthogonal to the third reference line RL3 in the concentric circle centered on the center point 7d of the support shaft 7b, and includes the second flow path 12 at a position corresponding to the second quadrant with respect to the third reference line RL3 and the fourth reference line RL4, and the rotation center C2 of the impeller 20 is located in the area A1 corresponding to the first quadrant or the area A3 corresponding to the third quadrant with respect to the third reference line RL3 and the fourth reference line RL4 (see FIG. 11), and the liquid delivery could be continuously performed with the direction of liquid flow being constant and the flow rate being also stable.

Thus, in the liquid delivery device 100 according to Example 2, when the drive motor 31 is arranged so that the rotation center C2 of the impeller 20 is located in the area A1 corresponding to the first quadrant or the area A3 corresponding to the third quadrant, and the impeller 20 can rotate in the rotation direction D1 (counterclockwise direction) in top-down view with the gap 24 being biased (that is, when the rotation center C3 of the drive motor 31 is arranged in the area A1 or the area A3), the liquid delivery could be continuously performed with the direction of liquid flow being constant in the forward flow F1 direction and the flow rate being also constant. Further, in Example 2, an average flow rate generally higher than that in Example 1 could be obtained for liquid delivery in the forward flow F1 direction.

Further, as shown in FIG. 17 and FIG. 14B, in the liquid delivery device 100 according to Example 2, the liquid delivery could be continuously performed with the direction of liquid flow being constant and the flow rate being also stable at the lower right S5 position of the liquid delivery chamber 7 (asymmetry).

That is, in the liquid delivery device 100, the liquid delivery chamber 7 includes the first flow path 11 at a position including the boundary line between the first quadrant and the fourth quadrant with respect to the third reference line RL3 passing through the center point 7d and parallel to the inflow direction of the liquid to the liquid delivery chamber 7 and the fourth reference line RL4 passing through the center point 7d and orthogonal to the third reference line RL3 in the concentric circle centered on the center point 7d of the support shaft 7b, and includes the second flow path 12 at a position corresponding to the second quadrant with respect to the third reference line RL3 and the fourth reference line RL4, and the rotation center C2 of the impeller 20 is located in the area A4 corresponding to the fourth quadrant (see FIG. 11), and the liquid delivery could be continuously performed with the direction of liquid flow being constant and the flow rate being also stable.

Thus, in the liquid delivery device 100 according to Example 2, when the drive motor 31 is arranged so that the rotation center C2 of the impeller 20 is located in the area A4 corresponding to the fourth quadrant, and the impeller 20 can rotate in the rotation direction D1 (counterclockwise direction) in top-down view with the gap 24 being biased (that is, when the rotation center C3 of the drive motor 31 is arranged in the area A4), the liquid delivery could be continuously performed with the direction of liquid flow being constant in the reverse flow F2 direction and the flow rate being also constant. Further, in Example 2, an average flow rate generally higher than that in Example 1 could be obtained for liquid delivery in the reverse flow F2 direction.

As shown in FIG. 18 and FIG. 14C, in the liquid delivery device 100 according to Example 3, the first flow path 11 and the second flow path 12 are formed on the same straight line via the liquid delivery chamber 7.

The liquid delivery device 100 according to Example 3 was able to continuously deliver liquid with a constant flow direction and a stable flow rate at the upper right S2 and lower left S4 positions of the liquid delivery chamber 7 (line symmetry Ver. 1).

That is, in the liquid delivery device 100, the liquid delivery chamber 7 includes the first flow path 11 at a position corresponding to the first quadrant with respect to the third reference line RL3 passing through the center point 7d and parallel to the inflow direction of the liquid to the liquid delivery chamber 7 and the fourth reference line RL4 passing through the center point 7d and orthogonal to the third reference line RL3 in the concentric circle centered on the center point 7d of the support shaft 7b, and includes the second flow path 12 at a position corresponding to the second quadrant with respect to the third reference line RL3 and the fourth reference line RL4, and the rotation center C2 of the impeller 20 is located in the area A1 corresponding to the first quadrant or the area A3 corresponding to the third quadrant with respect to the third reference line RL3 and the fourth reference line RL4 (see FIG. 12), and the liquid delivery could be continuously performed with the direction of liquid flow being constant and the flow rate being also stable.

Thus, in the liquid delivery device 100 according to Example 3, when the drive motor 31 is arranged so that the rotation center C2 of the impeller 20 is located in the area A1 corresponding to the first quadrant or the area A3 corresponding to the third quadrant, and the impeller 20 can rotate in the rotation direction D1 (counterclockwise direction) in top-down view with the gap 24 being biased (that is, when the rotation center C3 of the drive motor 31 is arranged in the area A1 or the area A3), the liquid delivery could be continuously performed with the direction of liquid flow being constant in the forward flow F1 direction and the flow rate being also constant. Further, in Example 3, an average flow rate generally higher than that in Example 1 could be obtained for liquid delivery in the forward flow F1 direction.

Further, as shown in FIG. 18 and FIG. 14C, in the liquid delivery device 100 according to Example 3, the liquid delivery could be continuously performed with the direction of liquid flow being constant and the flow rate being also stable at the upper left S3 and lower right S5 positions of the liquid delivery chamber 7 (line symmetry Ver. 1).

That is, in the liquid delivery device 100, the liquid delivery chamber 7 includes the first flow path 11 at a position corresponding to the first quadrant with respect to the third reference line RL3 passing through the center point 7d and parallel to the inflow direction of the liquid to the liquid delivery chamber 7 and the fourth reference line RL4 passing through the center point 7d and orthogonal to the third reference line RL3 in the concentric circle centered on the center point 7d of the support shaft 7b, and includes the second flow path 12 at a position corresponding to the second quadrant with respect to the third reference line RL3 and the fourth reference line RL4, and the rotation center C2 of the impeller 20 is located in the area A2 corresponding to the second quadrant or the area A4 corresponding to the fourth quadrant with respect to the third reference line RL3 and the fourth reference line RL4 (see FIG. 12), and the liquid delivery could be continuously performed with the direction of liquid flow being constant and the flow rate being also stable.

Thus, in the liquid delivery device 100 according to Example 3, when the drive motor 31 is arranged so that the rotation center C2 of the impeller 20 is located in the area A2 corresponding to the second quadrant or the area A4 corresponding to the fourth quadrant, and the impeller 20 can rotate in the rotation direction D1 (counterclockwise direction) in top-down view with the gap 24 being biased (that is, when the rotation center C3 of the drive motor 31 is arranged in the area A2 or the area A4), the liquid delivery could be continuously performed with the direction of liquid flow being constant in the reverse flow F2 direction and the flow rate being also constant. Further, in Example 3, an average flow rate generally higher than that in Example 1 could be obtained for liquid delivery in the reverse flow F2 direction.

As shown in FIG. 19 and FIG. 14D, in the liquid delivery device 100 according to Example 4, the first flow path 11 and the second flow path 12 are provided so that the inflow direction of the liquid flowing into the liquid delivery chamber 7 and the outflow direction of the liquid flowing out of the liquid delivery chamber 7 are aligned continuously by the arc of the first wall portion 7e.

The liquid delivery device 100 according to Example 4 was able to continuously deliver liquid with a constant flow direction and a stable flow rate at the top S6, upper left S9, and upper right S10 positions of the liquid delivery chamber 7 (line symmetry Ver. 2).

That is, in the liquid delivery device 100, the area of the liquid delivery chamber 7 is bisected by the second reference line RL2 passing through the center point 7d of the support shaft 7b and orthogonal to the first reference line RL1 passing through the center point 7d and the point P5 bisecting the length of the first wall portion 7e, and the liquid delivery could be continuously performed with the direction of liquid flow being constant and the flow rate being also stable in the semicircular area A5 including the first wall portion 7e (see FIG. 13).

Thus, in the liquid delivery device 100 according to Example 4, when the drive motor 31 is arranged so that the rotation center C2 of the impeller 20 is located in the semicircular area A5, and the impeller 20 can rotate in the rotation direction D1 (counterclockwise direction) in top-down view with the gap 24 being biased (that is, when the rotation center C3 of the drive motor 31 is arranged in the semicircular area A5), the liquid delivery could be continuously performed with the direction of liquid flow being constant in the forward flow F1 direction and the flow rate being also constant. Further, in Example 4, an average flow rate generally higher than that in Example 1 could be obtained for liquid delivery in the forward flow F1 direction.

Since the liquid delivery device 100 according to Example 4 not only has a high flow rate but also has a large area where the drive motor 31 can be installed (a wide allowable range for installing the drive motor 31), it can be said to be a more preferred aspect.

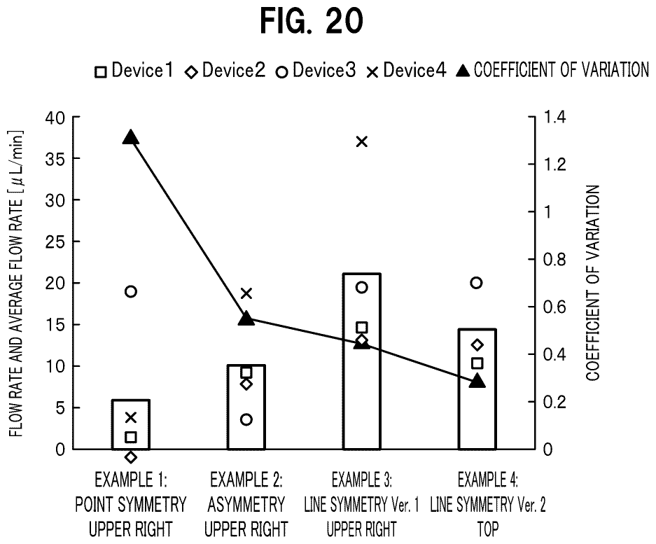

FIG. 20 is a graph showing the flow rate, average flow rate, and coefficient of variation of each device used in the liquid delivery devices 100 according to Examples 1 to 4 after 45 minutes from the start of driving the drive motor 31. FIG. 20 shows the flow rate when the drive motor 31 is arranged at the position where the average flow rate is highest in the liquid delivery in the forward flow F1 direction in each of the examples shown in FIGS. 16 to 19. That is, in Examples 1 to 3, the flow rate is when the drive motor 31 is arranged at the upper right S2 position in the liquid delivery chamber 7, and in Example 4, the flow rate is when the drive motor 31 is arranged at the top S6 position in the liquid delivery chamber 7. The number of devices used in each example is four (Device 1 to 4) in Examples 1 to 3 and three (Device 1 to 3) in Example 4. In FIG. 20, the left vertical axis represents the flow rate and the average flow rate [μL/min], and the right vertical axis represents the coefficient of variation. Further, in FIG. 20, “n” represents the flow rate of Device 1 in each example, “O” represents the flow rate of Device 2 in each example, “●” represents the flow rate of Device 3 in each example, and “x” represents the flow rate of Device 4 in each example. The vertical bar of each example indicates the average flow rate. “A” indicates the coefficient of variation for the flow rates of Device 1 to 4 in Examples 1 to 3 and the coefficient of variation for the flow rates of Device 1 to 3 in Example 4.

As shown in FIG. 20, the liquid delivery device 100 according to Example 3 had the highest average flow rate, followed by the liquid delivery device 100 according to Example 4. The liquid delivery device 100 according to Example 4 had the lowest coefficient of variation and a stable flow rate. From this, it was found that the shape of the liquid delivery chamber 7 in Examples 3 and 4, that is, the shape in which the first flow path 11 and the second flow path 12 are provided at line-symmetrical positions with respect to the support shaft 7b of the liquid delivery chamber 7 (see FIGS. 12, 13, 14C, and 14D), and further, the drive motor 31 is arranged so that the rotation center C2 of the impeller 20 is located in a predetermined area and the impeller 20 can rotate with the gap 24 formed between the outer periphery 7c of the support shaft 7b and the inner circle 21a of the annular portion 21 being biased is more preferable.

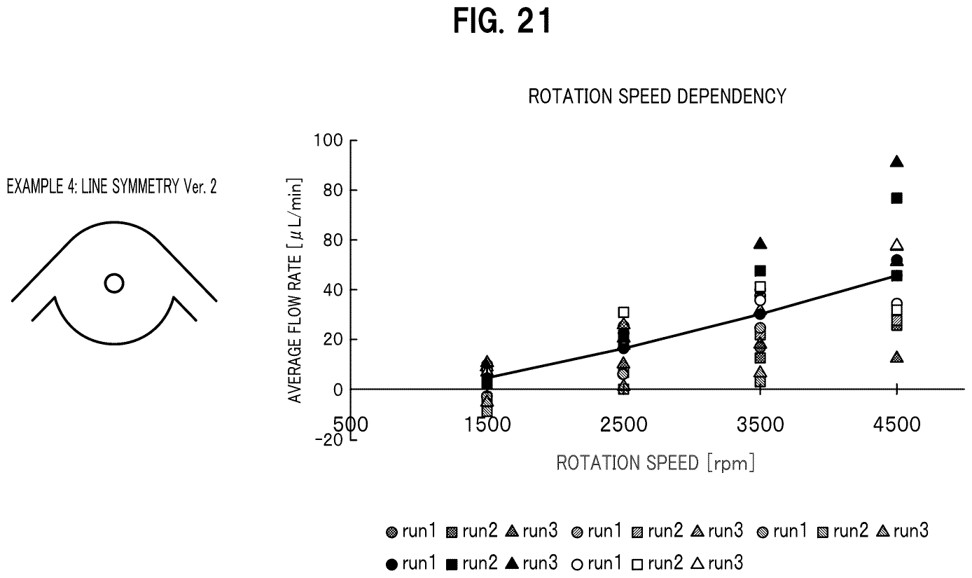

FIG. 21 is a graph showing that the average flow rate of the liquid delivery device 100 has rotation speed dependency. The shape of the liquid delivery chamber 7 in Example 4 is shown on the left side of FIG. 21. FIG. 21 shows the results of investigating the average flow rate at the respective rotation speeds by using the liquid delivery device 100 according to Example 4, adjusting the rotation speed of the drive motor 31 to adjust the rotation speed of the impeller 20 to about 1500 rpm, about 2500 rpm, about 3500 rpm, and about 4500 rpm, respectively. In FIG. 21, the horizontal axis represents the rotation speed [rpm], and the vertical axis represents the average flow rate [μL/min]. In conducting this investigation, five liquid delivery devices 100 each having a liquid delivery chamber 7 self-made based on the same design drawing were prepared and used. The flow rate was measured three times for each device (runs 1 to 3). The drive motor 31 was arranged so that the rotation center C3 thereof was located at the top S6 position in the liquid delivery chamber 7 where the average flow rate was highest (see FIG. 14D).

As shown in FIG. 21, it was found that in the liquid delivery device 100 according to Example 4, the average flow rate increases as the rotation speed of the impeller 20 increases. That is, it was found that the average flow rate of the liquid delivery device 100 has rotation speed dependency.

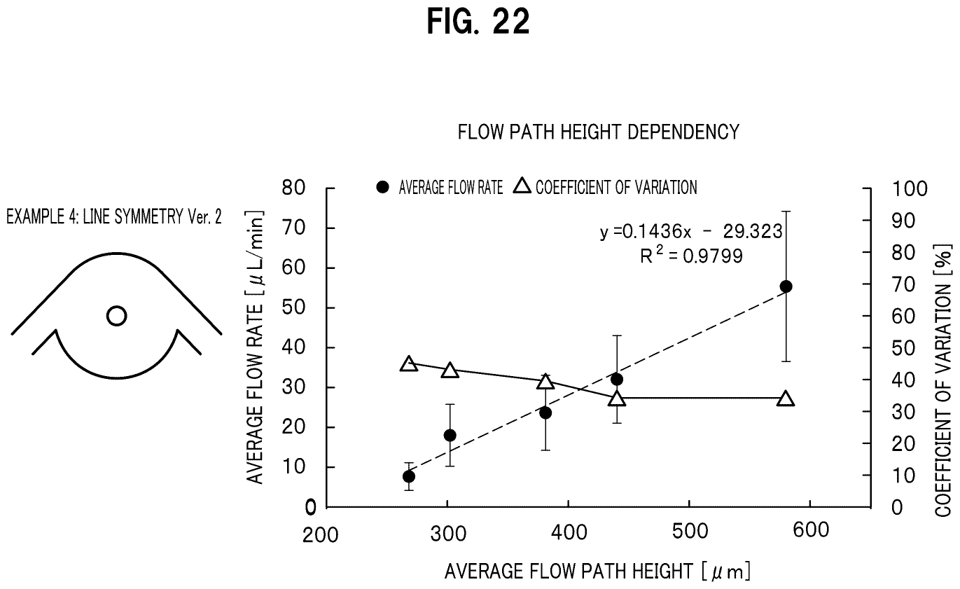

FIG. 22 is a graph showing that the average flow rate of the liquid delivery device 100 according to Example 4 in FIG. 14D has flow path depth dependency. The shape of the liquid delivery chamber 7 in Example 4 is shown on the left side of FIG. 22. FIG. 22 shows the results of investigating the average flow rate when the average flow path depth of the liquid delivery chamber 7, the first flow path 11, the second flow path 12, and the return flow path 13 was adjusted to 268.0 μm, 302.0 μm, 380.0 μm, 440.0 μm, or 580.0 μm to configure each of them similarly to the liquid delivery device 100 according to Example 4 and the rotation speed of the impeller 20 was adjusted to about 2500 rpm. In FIG. 22, the horizontal axis represents the average flow path depth [μm], the left vertical axis represents the average flow rate [μL/min], and the right vertical axis represents the coefficient of variation [%]. “●” represents the average flow rate at each average flow path depth. “Δ” represents the coefficient of variation at each average flow path depth. In conducting this investigation, three liquid delivery devices 100 were prepared for each flow path depth. The flow rate was measured three times for each device (runs 1 to 3). The error bar of each average flow rate in FIG. 22 indicates the standard deviation (SD) between devices based on nine data obtained by performing runs 1 to 3 for each of these three devices. The drive motor 31 was arranged so that the rotation center C3 thereof was located at the top S6 position in the liquid delivery chamber 7 where the average flow rate was highest (see FIG. 14D).

As shown in FIG. 22, it was found that the average flow rate increases as the average flow path depth of the liquid delivery device 100 according to Example 4 increases. Further, it was found that the coefficient of variation tends to decrease as the average flow path depth increases. From this, it was found that the stability of the liquid delivery performance changes depending on the change in the average flow path depth.

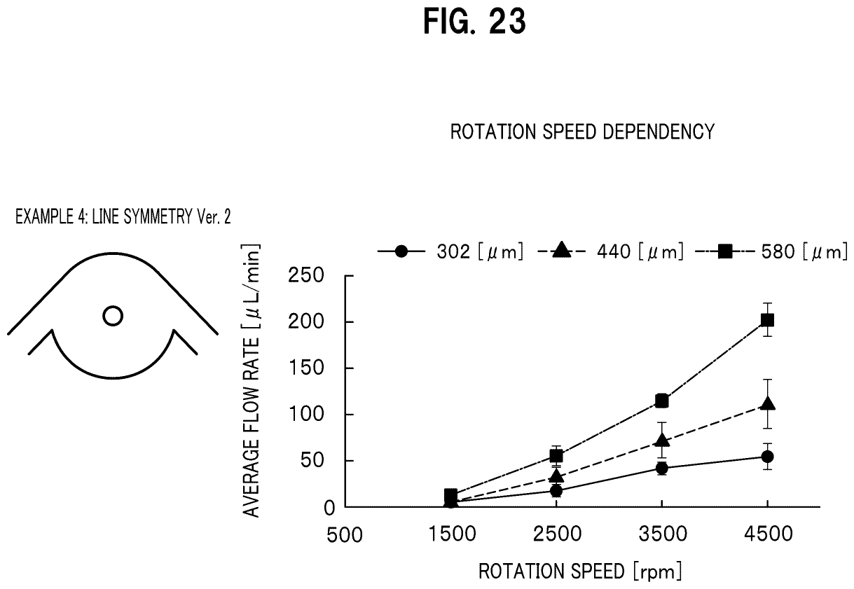

FIG. 23 is a graph showing that the average flow rate of the liquid delivery devices 100 according to Example 4 with different average flow path depths in FIG. 14D has rotation speed dependency. The shape of the liquid delivery chamber 7 in Example 4 is shown on the left side of FIG. 23. FIG. 23 shows the results of investigating the average flow rate at the respective rotation speeds by using the liquid delivery devices 100 having average flow path depths of 302.0 μm, 440.0 μm, and 580.0 μm used in the example of FIG. 22, adjusting the rotation speed of the drive motor 31 to adjust the rotation speed of the impeller 20 to about 1500 rpm, about 2500 rpm, about 3500 rpm, and about 4500 rpm, respectively. In FIG. 23, the horizontal axis represents the rotation speed [rpm], and the vertical axis represents the average flow rate [μL/min]. “●” indicates the average flow rate when the average flow path depth is 302.0 μm. “▴” indicates the average flow rate when the average flow path depth is 440.0 μm. “□” indicates the average flow rate when the average flow path depth is 580.0 μm. In conducting this investigation, three liquid delivery devices 100 were prepared and used for each flow path depth. The flow rate was measured three times for each device (runs 1 to 3). The error bar of each average flow rate in FIG. 23 indicates the standard deviation (SD) between devices based on nine data obtained by performing runs 1 to 3 for each of these three devices. The drive motor 31 was arranged so that the rotation center C3 thereof was located at the top S6 position in the liquid delivery chamber 7 where the average flow rate was highest (see FIG. 14D).

As shown in FIG. 23, it was found that regardless of the average flow path depth of the liquid delivery device 100 according to Example 4, the average flow rate increases as the rotation speed of the impeller 20 increases. Further, similarly to FIG. 22, it was proved that the average flow rate is higher in the order of the average flow path depth regardless of the rotation speed.

Here, using the shape of the liquid delivery chamber 7 in Example 4, it has been shown that the average flow rate has average flow path depth dependency and that the average flow rate has rotation speed dependency even when the average flow path depths are different, but the same effects can be obtained not only with the shape of the liquid delivery chamber 7 in Example 4 but also with the shape of the liquid delivery chamber 7 in each of Examples 1 to 3.

Further, in the liquid delivery devices 100 according to Examples 1, 3, and 4 (the liquid delivery devices 100 in which the first flow path 11 and the second flow path 12 are point-symmetrical or line-symmetrical with respect to the support shaft 7b of the liquid delivery chamber 7), the liquid flow when the rotation direction of the drive motor 31 and the rotation direction of the impeller 20 rotated thereby are the rotation direction D2 (clockwise direction) was confirmed in the same manner as described above.

Specifically, the rotation center C3 of the drive motor 31 was arranged in the area A1 of the liquid delivery device 100 according to Example 1, and the impeller 20 was rotated in the rotation direction D2 (clockwise direction).

The rotation center C3 of the drive motor 31 was arranged in the area A1 or the area A3 of the liquid delivery device 100 according to Example 3, and the impeller 20 was rotated in the rotation direction D2 (clockwise direction).

The rotation center C3 of the drive motor 31 was arranged in the semicircular area A5 of the liquid delivery device 100 according to Example 4, and the impeller 20 was rotated in the rotation direction D2 (clockwise direction).

As a result, it was found that in all cases, the liquid could be constantly flowed in the reverse flow F2 direction, and the liquid delivery could be continuously performed with a stable flow rate. That is, it was confirmed that in the liquid delivery devices 100 according to Examples 1, 3, and 4, the liquid delivery direction can be adjusted by the rotation direction of the impeller 20.

FIG. 24 is an explanatory diagram showing the shape of the liquid delivery section 3 of the device body 10 and the arrangement position of the rotation center C3 of the drive motor 31, which will be described later, according to Example 5. As shown in FIG. 24, the liquid delivery section 3 of the device body 10 according to Example 5 has the same shape as the liquid delivery section 3 of the device body 10 according to Example 2 (see FIG. 14B). That is, as shown in FIG. 24, in the liquid delivery section 3 of the device body 10 according to Example 5, similarly to Example 2, the first flow path 11 and the second flow path 12 are asymmetrical with respect to the support shaft 7b of the liquid delivery chamber 7 (see FIGS. 11 and 14B).

Therefore, in the concentric circle centered on the center point 7d of the support shaft 7b, the liquid delivery chamber 7 of the device body 10 according to Example 5 includes the first flow path 11 at a position including the boundary line between the first quadrant and the fourth quadrant with respect to the third reference line RL3 passing through the center point 7d and parallel to the inflow direction of the liquid to the liquid delivery chamber 7 and the fourth reference line RL4 passing through the center point 7d and orthogonal to the third reference line RL3, and includes the second flow path 12 at a position corresponding to the second quadrant with respect to the third reference line RL3 and the fourth reference line RL4. The third reference line RL3 described above can be considered as, for example, the x-axis, and the fourth reference line RL4 described above can be considered as, for example, the y-axis.

In the device body 10 according to Example 5, the rotation center C3 of the drive motor 31 was arranged at four locations: upper right S2, upper left S3, lower left S4, and lower right S5, which are shown by “Q” in FIG. 24, the impeller 20 was rotated, and changes over time in the average flow rate were investigated.

For the changes over time in the average flow rate, a close-up camera was installed close to the XXIVb portion in FIG. 24, that is, the second flow path 12, and the state of the solution flowing through the second flow path 12 was continuously photographed. Then, the average flow rate of the liquid delivery device 100 according to Example 5 was calculated using PIV analysis software (Flow Expert 2D2C).

### This Investigation was Conducted Under the Following Conditions.

Solution: Fluorescent microbead dispersion solution with a diameter of about 1.0 μm
Measurement time: 30 seconds from 10 minutes after the start of driving the drive motor 31 (0 min)
Rotation speed: about 2200 rpm
Rotation direction: rotation direction D2 (clockwise direction) in top-down view

In the device body 10 according to Example 5, the rotation direction of the drive motor 31 and the rotation direction of the impeller 20 rotated thereby were the rotation direction D2 (clockwise direction), which is the reverse rotation direction to that in Example 2 and the like. That is, in the device body 10 according to Example 5, the impeller 20 rotates in the same direction as the outflow direction of the liquid flowing from the second flow path 12 to the first flow path 11 along the first wall portion 7e.

As a result, it was confirmed that the liquid flowed from the second flow path 12 to the first flow path 11 at the upper right S2, upper left S3, and lower right S5 positions. That is, in the device body 10 according to Example 5, by arranging the drive motor 31 so that the rotation center C2 of the impeller 20 is located in the area A1 corresponding to the first quadrant, the area A2 corresponding to the second quadrant, or the area A4 corresponding to the fourth quadrant, and the impeller 20 can rotate with the gap 24 being biased, it was confirmed that the liquid constantly flows from the second flow path 12 to the first flow path 11, and the flow rate is stable.

Thus, also when the first flow path 11 and the second flow path 12 are asymmetrical with respect to the support shaft 7b of the liquid delivery chamber 7, the device body 10 according to Example 5 can constantly flow the liquid in the reverse flow F2 direction by rotating the impeller 20 in the reverse direction, similarly to the case where the first flow path 11 and the second flow path 12 are point-symmetrical or line-symmetrical with respect to the support shaft 7b of the liquid delivery chamber 7, and it was found that the liquid delivery can be continuously performed with a stable flow rate.

Although the embodiments and examples of the present invention have been described above, the present invention is not limited to the above-described embodiments and examples, and each component described above can be appropriately changed without departing from the spirit of the present invention.

### Modification of Third Embodiment

For example, in the third embodiment, as shown in FIG. 12 and FIG. 14C, it has been described that the first flow path 11 and the second flow path 12 may be in direct contact with each other at the first connection portion P1 and the second connection portion P2 with respect to the circular liquid delivery chamber 7 in top-down view. That is, the third embodiment has described an aspect in which the wall portion of the circular liquid delivery chamber 7 and the wall portion of the first flow path 11 and the second flow path 12 that is outside the width W3 (see FIG. 5) direction of the liquid delivery chamber 7 are in contact with each other at one point. In the third embodiment, the length of the first wall portion 7e formed by the circular inner peripheral surface 7a between the first connection portion P1 and the second connection portion P2 is thereby set to 0. However, the present embodiment is not limited to this aspect.

Here, FIG. 25 is an explanatory diagram of the liquid delivery section 3 of the liquid delivery device 100 according to a modification of the third embodiment.

As shown in FIG. 25, in this modification, the first flow path 11 and the second flow path 12 formed on the same straight line can be arranged further inward in the width W3 direction of the circular liquid delivery chamber 7. In this case, the liquid delivery device 100 according to this modification has a first wall portion 7e, which is formed by a first connection portion P1 connected to the first flow path 11, a second connection portion P2 connected to the second flow path 12, and a circular inner peripheral surface 7a between the first connection portion P1 and the second connection portion P2 in top-down view. Further, the liquid delivery chamber 7 has a second wall portion 7f, which is formed by a third connection portion P3 connected to the first flow path 11, a fourth connection portion P4 connected to the second flow path 12, and a circular inner peripheral surface 7a between the third connection portion P3 and the fourth connection portion P4 in top-down view.

Even in the aspect according to such a modification, similarly to the third embodiment, liquid can be continuously delivered in a constant flow direction with a stable flow rate.

### Modification of Fourth Embodiment

Further, for example, in the fourth embodiment, as shown in FIG. 13 and FIG. 14D, with respect to the circular liquid delivery chamber 7 in top-down view, the first flow path 11 and the second flow path 12 are provided so that the inflow direction of the liquid flowing into the liquid delivery chamber 7 and the outflow direction of the liquid flowing out of the liquid delivery chamber 7 are aligned continuously by the arc of the first wall portion 7e, and it has been described that the angle formed by the first flow path 11 and the second flow path 12 is, for example, 90°. However, the present embodiment is not limited to this aspect.

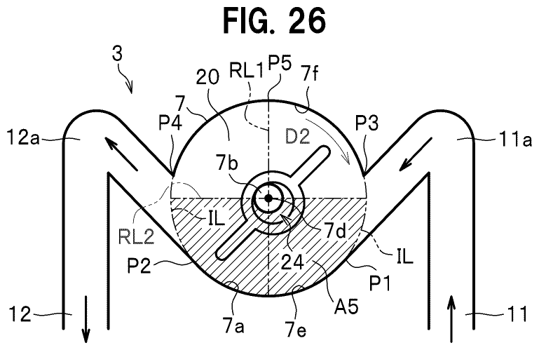

Here, FIG. 26 is an explanatory diagram of the liquid delivery section 3 of the liquid delivery device 100 according to a modification of the fourth embodiment.

In the fourth embodiment, as shown in FIG. 13 and FIG. 14D, the first flow path 11, the liquid delivery chamber 7, and the second flow path 12 are formed in an A shape, but as shown in FIG. 26, they can also be formed in an M shape.

In this modification, the first flow path 11 has a first folded portion 11a formed by folding back at an arbitrary position between the liquid delivery chamber 7 and the not-shown first reservoir 5. The second flow path 12 has a second folded portion 12a formed by folding back at an arbitrary position between the liquid delivery chamber 7 and the not-shown second reservoir 6. Therefore, in this modification, the degree of freedom in design of the liquid delivery device 100 can be improved.

As shown in FIG. 26, in the liquid delivery chamber 7 of this modification, similarly to the fourth embodiment, a length of a first wall portion 7e, which is formed by a first connection portion P1 connected to the first flow path 11, a second connection portion P2 connected to the second flow path 12, and a circular inner peripheral surface 7a between the first connection portion P1 and the second connection portion P2 in top-down view is shorter than a length of a second wall portion 7f, which is formed by a third connection portion P3 connected to the first flow path 11, a fourth connection portion P4 connected to the second flow path 12, and a circular inner peripheral surface 7a between the third connection portion P3 and the fourth connection portion P4.

Further, in this modification, the first flow path 11 and the second flow path 12 are provided so that the inflow direction of the liquid flowing into the liquid delivery chamber 7 and the outflow direction of the liquid flowing out of the liquid delivery chamber 7 are aligned continuously by the arc of the first wall portion 7e. Also in this modification, similarly to the fourth embodiment, the angle formed by the first flow path 11 and the second flow path 12 can be, for example, 90°.

That is, in this modification, as shown in FIG. 26, the first flow path 11 and the second flow path 12 are provided at positions that are line-symmetrical with respect to the support shaft 7b of the liquid delivery chamber 7 (with respect to the first reference line RL1, which will be described later).

Also in this modification, as an example, the impeller 20 can be rotated in the same direction as the outflow direction of the liquid flowing along the first wall portion 7e. That is, also in this modification, as an example, the impeller 20 can be rotated in the direction from the first flow path 11 to the second flow path 12 along the first wall portion 7e. In this modification, the impeller 20 rotates in the rotation direction D2 (clockwise direction) in top-down view.

In this modification, as shown in FIG. 26, the drive motor 31 is arranged so that the area is bisected by the second reference line RL2 passing through the center point 7d of the support shaft 7b and orthogonal to the first reference line RL1 passing through the center point 7d and the point P5 bisecting the length of the first wall portion 7e, and the rotation center C2 of the impeller 20 is located in the semicircular area A5 including the first wall portion 7e, and the impeller 20 can rotate with the gap 24 being biased. The second reference line RL2 described above can be considered as, for example, the x-axis, and the first reference line RL1 described above can be considered as, for example, the y-axis.

Even in the aspect according to such a modification, similarly to the fourth embodiment, liquid can be continuously delivered in a constant flow direction with a stable flow rate.

In this modification, by rotating the impeller 20 in the rotation direction D1 (counterclockwise direction, see FIG. 5) in top-down view, liquid can be continuously delivered from the second flow path 12 to the first flow path 11 in a constant flow direction with a stable flow rate.

### (Other Modifications)

Further, for example, the center point 7d of the support shaft 7b and the rotation center C3 of the drive motor 31 only need to be relatively offset from each other. For example, the formation position of the support shaft 7b may be formed in the areas A1 to A5 described in the above embodiments and examples, and the rotation center C3 of the drive motor 31 may be set to the same position as in the centering method, which is the conventional method, that is, the same position as the position of the central axis C1 of the liquid delivery chamber 7. Even in this case, the impeller 20 comes into contact with the support shaft 7b at a point, so that the point contact method can be implemented. Therefore, even in such an aspect, similarly to the first to fourth embodiments, liquid can be continuously delivered in a constant flow direction with a stable flow rate.

### Sixth Embodiment

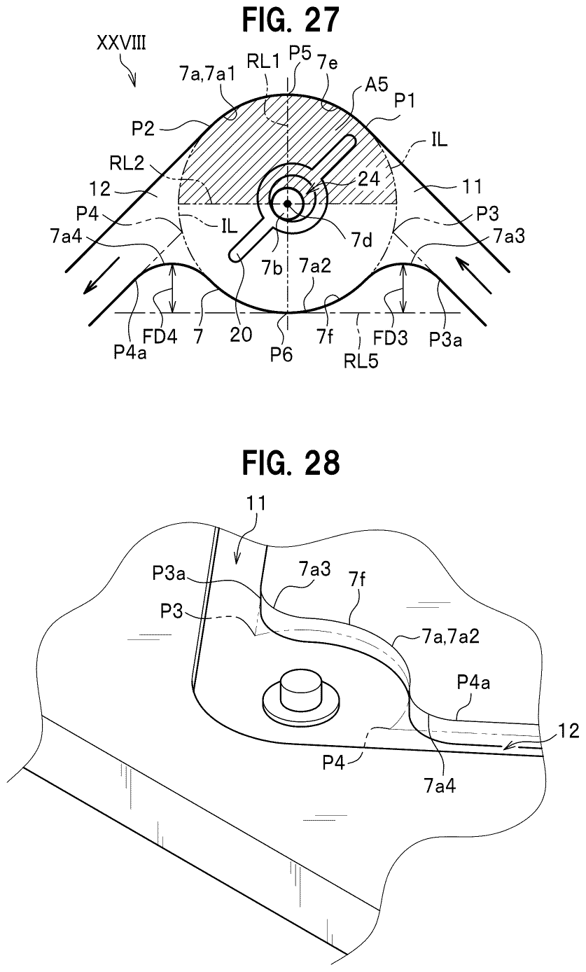

FIG. 27 is an explanatory diagram of the liquid delivery section 3 of the liquid delivery device 100 according to the sixth embodiment. FIG. 28 is a perspective view seen from the direction of arrow XXVIII in FIG. 27.

As shown in FIG. 27, the liquid delivery chamber 7 of the liquid delivery section 3 in the sixth embodiment also has a circular shape in top-down view.

Further, in this aspect, the liquid delivery chamber 7 has a first wall portion 7e and a second wall portion 7f.

The first wall portion 7e is formed by a first connection portion P1 connected to the first flow path 11, a second connection portion P2 connected to the second flow path 12, and a first inner peripheral surface 7al forming a circular arc between the first connection portion P1 and the second connection portion P2 in top-down view.

The second wall portion 7f is formed by a third connection portion P3a connected to the first flow path 11, a fourth connection portion P4a connected to the second flow path 12, a second inner peripheral surface 7a2 forming a circular arc between the third connection portion P3a and the fourth connection portion P4a, a third inner peripheral surface 7a3 having an arc shape and a curvature opposite to that of the second inner peripheral surface 7a2 between the third connection portion P3a and the second inner peripheral surface 7a2, and a fourth inner peripheral surface 7a4 having an arc shape and a curvature opposite to that of the second inner peripheral surface 7a2 between the fourth connection portion P4a and the second inner peripheral surface 7a2.

Further, in this aspect, the first flow path 11 and the second flow path 12 are provided so that the inflow direction of the liquid flowing into the liquid delivery chamber 7 and the outflow direction of the liquid flowing out of the liquid delivery chamber 7 are aligned continuously by the arc of the first wall portion 7e. The angle formed by the first flow path 11 and the second flow path 12 can be, for example, 90°.

That is, in the sixth embodiment, as shown in FIG. 27, the first flow path 11 and the second flow path 12 are provided at positions that are line-symmetrical with respect to the support shaft 7b of the liquid delivery chamber 7 (with respect to the first reference line RL1, which will be described later).

In this aspect, as an example, the impeller 20 can be rotated in the same direction as the outflow direction of the liquid flowing along the first wall portion 7e. That is, in this aspect, as an example, the impeller 20 can be rotated in the direction from the first flow path 11 to the second flow path 12 along the first wall portion 7e.

In this aspect, as shown in FIG. 27, the drive motor 31 is arranged so that the area is bisected by the second reference line RL2 passing through the center point 7d of the support shaft 7b and orthogonal to the first reference line RL1 passing through the center point 7d and the point P5 bisecting the length of the first wall portion 7e, and the rotation center C2 of the impeller 20 (see FIGS. 5 and 6B) is located in the semicircular area A5 including the first wall portion 7e, and the impeller 20 can rotate with the gap 24 being biased. The second reference line RL2 described above can be considered as, for example, the x-axis, and the first reference line RL1 described above can be considered as, for example, the y-axis.

In the sixth embodiment, since the drive motor 31 is arranged so that the rotation center C2 of the impeller 20 is located in the semicircular area A5 and the impeller 20 can rotate with the gap 24 being biased, the rotation (rotation position) of the impeller 20 is stabilized. Therefore, the liquid delivery device 100 according to the sixth embodiment can continuously deliver liquid in a constant flow direction with a stable flow rate.

Further, as shown in FIGS. 27 and 28, in the liquid delivery device 100 according to the sixth embodiment, the second wall portion 7f is connected to the first flow path 11 by the third inner peripheral surface 7a3, and is connected to the second flow path 12 by the fourth inner peripheral surface 7a4. Therefore, compared to the liquid delivery device 100 according to the fourth embodiment in which the third connection portion P3 (see FIGS. 13 and 28) and the fourth connection portion P4 (see FIGS. 13 and 28) are formed at an acute angle, the liquid delivery device 100 according to the sixth embodiment is less likely to be chipped or the like at these connection portions during manufacturing, and the flow paths are easy to form. Therefore, the device is easy to manufacture.

Further, as shown in FIG. 27, in the sixth embodiment, taking, as a reference, the fifth reference line RL5 that passes through the intersection P6 between the second inner peripheral surface 7a2 and the first reference line RL1 and is parallel to the second reference line RL2, formation dimensions FD3 and FD4 of the third inner peripheral surface 7a3 and the fourth inner peripheral surface 7a4, measured perpendicular to the fifth reference line RL5 (that is, parallel to the first reference line RL1) at longest points thereof, can both be set the same. In this case, the liquid delivery device 100 can be used in the same manner in both the forward flow F1 and the reverse flow F2.

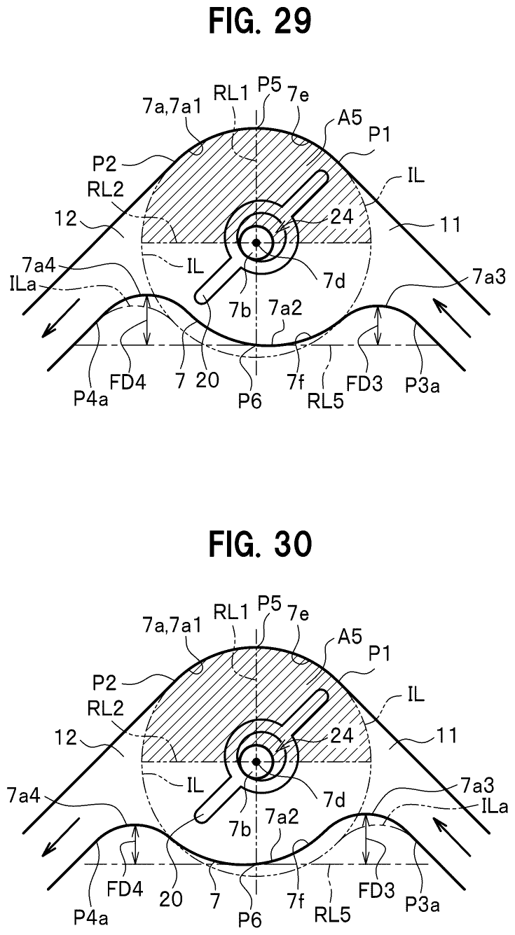

FIGS. 29 and 30 are explanatory diagrams of the liquid delivery section 3 of the liquid delivery device 100 according to a modification of the sixth embodiment. In FIGS. 29 and 30, for comparison with FIG. 28, the second wall portion 7f (the second inner peripheral surface 7a2, the third inner peripheral surface 7a3, and the fourth inner peripheral surface 7a4) shown in FIG. 28 is indicated by a virtual line ILa.

As shown in FIGS. 29 and 30, in the modification of the sixth embodiment, taking, as a reference, the fifth reference line RL5 that passes through the intersection P6 between the second inner peripheral surface 7a2 and the first reference line RL1 and is parallel to the second reference line RL2, formation dimensions FD3 and FD4 of the third inner peripheral surface 7a3 and the fourth inner peripheral surface 7a4, measured perpendicular to the fifth reference line RL5 (that is, parallel to the first reference line RL1) at longest points thereof, may be different from each other. In this case, the degree of freedom in design of the liquid delivery device 100 can be improved.

Further, in this case, it is preferable to shorten the formation dimension of the third inner peripheral surface 7a3 and the fourth inner peripheral surface 7a4 that is arranged on the liquid outflow side in the liquid delivery chamber 7. By doing so, when the side with the shortened formation dimension is set as the outflow side, a larger flow rate can be obtained.

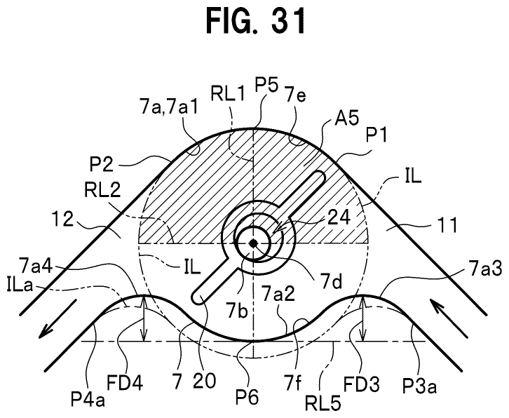

FIG. 31 is an explanatory diagram of the liquid delivery section 3 of the liquid delivery device 100 according to a further modification of the sixth embodiment. In FIG. 31, for comparison with FIG. 28, the second wall portion 7f (the second inner peripheral surface 7a2, the third inner peripheral surface 7a3, and the fourth inner peripheral surface 7a4) shown in FIG. 28 is indicated by a virtual line ILa.

As shown in FIG. 31, in the further modification of the sixth embodiment, the second wall portion 7f (the second inner peripheral surface 7a2, the third inner peripheral surface 7a3, and the fourth inner peripheral surface 7a4) is moved closer to the second reference line RL2 as compared to the liquid delivery device 100 according to the sixth embodiment shown in FIG. 27. The second wall portion 7f in this modification can be provided close to the impeller 20 so as not to contact the rotation trajectory 23 (see FIG. 5) of the impeller 20. Also in this case, the liquid delivery device 100 according to the further modification of the sixth embodiment can continuously deliver liquid in a constant flow direction with a stable flow rate.

The arc shape or arc may be a case where the arcs of the inner peripheral surfaces facing each other at the first flow path 11 and the second flow path 12 have the same curvature (for example, FIG. 27) or different curvatures (for example, FIGS. 29 to 31).

### Example 2

### Second Example

### (Study 2 on the Shape of the Liquid Delivery Section 3 of the Device Body 10)

Similarly to [First Example], a device body 10 made of polystyrene and having a rectangular shape in top-down view, which is formed in a block shape, was prepared. [Second Example] is different from [First Example] in that the polystyrene device body 10 is cut with a three-dimensional plotter to form a liquid flow structure 1 (a liquid flow section 2, a liquid delivery section 3, and a loop-shaped flow path 4) as shown in FIG. 4.

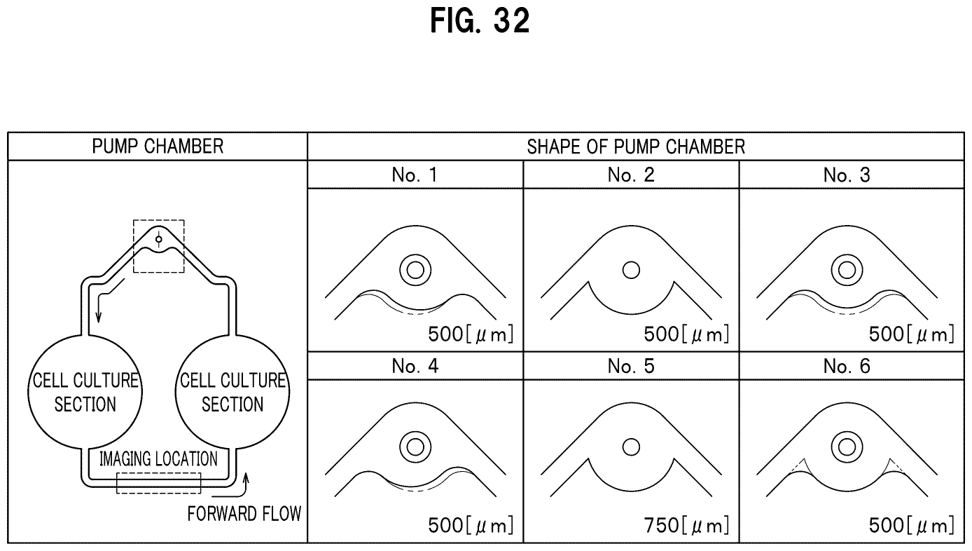

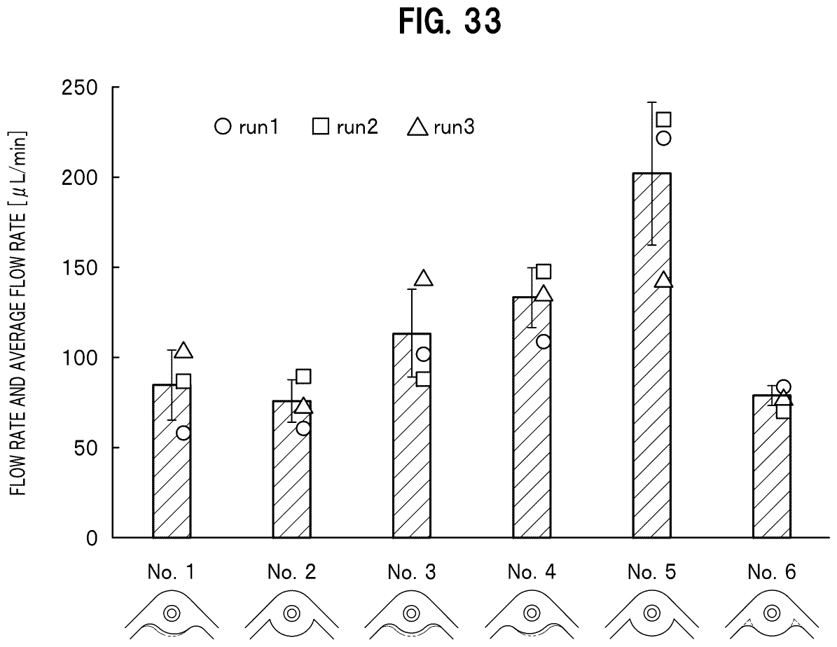

Further, device bodies 10 each having a pump chamber (liquid delivery section 3) having a shape shown in No. 1 to No. 6 of FIG. 32 were prepared, and the liquid flow rate was measured. The liquid flow rate was measured at the “imaging location” shown in FIG. 32. FIG. 32 is an explanatory diagram showing the shapes of the liquid delivery sections 3 according to Nos. 1 to 6 that were adopted when investigating how the difference in the shape of the liquid delivery section 3 of the device body 10 affects the liquid flow rate.

Here, No. 2 corresponds to Example 4 of [First Example], and the depth dimension of the loop-shaped flow path 4 and the liquid delivery chamber 7 is 500 μm.

No. 5 has the same shape of the liquid delivery section 3 (liquid delivery chamber 7) as Example 4 of [First Example], but the depth dimension of the loop-shaped flow path 4 and the liquid delivery chamber 7 is 750 μm.

No. 6 is obtained by respectively shaving the third connection portion P3 and the fourth connection portion P4 in Example 4 of [First Example] to round the shapes thereof and forming the arc-shaped third inner peripheral surface 7a3 having a curvature opposite to that of the second inner peripheral surface 7a2 and the arc-shaped fourth inner peripheral surface 7a4 having a curvature opposite to that of the second inner peripheral surface 7a2.

No. 1 is obtained by setting the formation dimension FD3 on the liquid inflow side to be the same as that of No. 6 and lengthening the formation dimension FD4 on the liquid outflow side.

No. 4 is obtained by lengthening the formation dimension FD3 on the liquid inflow side and setting the formation dimension FD4 on the liquid outflow side to be the same as that of No. 6.

No. 3 is obtained by lengthening the formation dimension FD3 on the liquid inflow side and the formation dimension FD4 on the liquid outflow side with respect to No. 6. The formation dimension FD3 and the formation dimension FD4 in No. 3 have the same length. The second wall portion 7f in No. 3 is generally closer to the first wall portion 7e.

FIG. 33 is a graph showing individual flow rates and average flow rates [μL/min] when No. 1 to No. 6 were measured three times each. ∘ indicates the flow rate of run 1, □ indicates the flow rate of run 2, and Δ indicates the flow rate of run 3, and the bar graph indicates the average flow rate. The error bar indicates the variation (inter-experimental variation) in the flow rate of the three experiments (runs 1 to 3) performed using the same device, that is, the standard deviation. The experiment regarding FIG. 33 was conducted under the experimental conditions shown in Table 1.

TABLE 1

| | |
|---|---|
| Pump Chamber | Nos. 1 to 6 |
| Drive Motor | 1ch Motor Manufactured by mfsworks |
| | (Counterclockwise) |
| Rotation Speed | 4500 [rpm] |
| Perfusion Time | 15 [min] × Number of Runs, 3 |

As shown in FIG. 33, when the flow path depth was the same and the rotation was counterclockwise, the liquid flow rates of No. 3 and No. 4 were large.

There was no significant difference in the liquid flow rate between No. 2 corresponding to Example 4 of [First Example] and No. 6 in which the third connection portion P3 and the fourth connection portion P4 are rounded with respect to No. 2 (the arc-shaped third inner peripheral surface 7a3 having a curvature opposite to that of the second inner peripheral surface 7a2 and the arc-shaped fourth inner peripheral surface 7a4 having a curvature opposite to that of the second inner peripheral surface 7a2 are formed).

As can be seen from a comparison between No. 1 and No. 4, the liquid flow rate is larger when the formation dimension FD4 on the liquid outflow side is shorter (No. 4).

Since the flow path depth of No. 5 is large, the flow rate thereof is largest.

### Example 3

### Third Example

### (Study 3 on the Shape of the Liquid Delivery Section 3 of the Device Body 10)

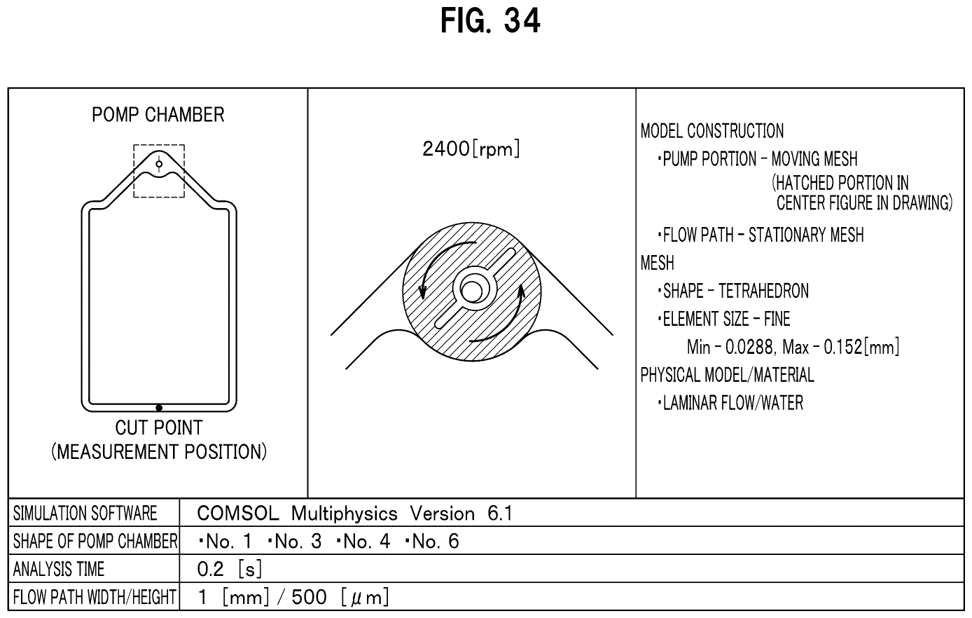

Next, the shape of the liquid delivery section 3 of the device body 10 was studied by simulation. Here, the influence of the difference in the shape of the pump chamber (liquid delivery section 3) on the liquid flow rate was investigated. FIG. 34 is an explanatory diagram showing and explaining the conditions, etc., of the simulation for examining the shape of the liquid delivery section 3 of the device body 10.

The experimental conditions of the simulation are as shown in the table at the bottom of FIG. 34, and the simulation software used is COMSOL Multiphysics Version 6.1. The shapes of the pump chambers (liquid delivery sections 3) are No. 1, No. 3, No. 4, and No. 6. The analysis time was 0.2 seconds. The flow path width was 1 mm, and the flow path depth was 500 μm.

Further, as shown in FIG. 34, in the simulation, a pump chamber is provided in a part of the flow path, and a cut point (measurement position) is provided in another part thereof. The rotation speed of the impeller 20 was 2400 rpm.

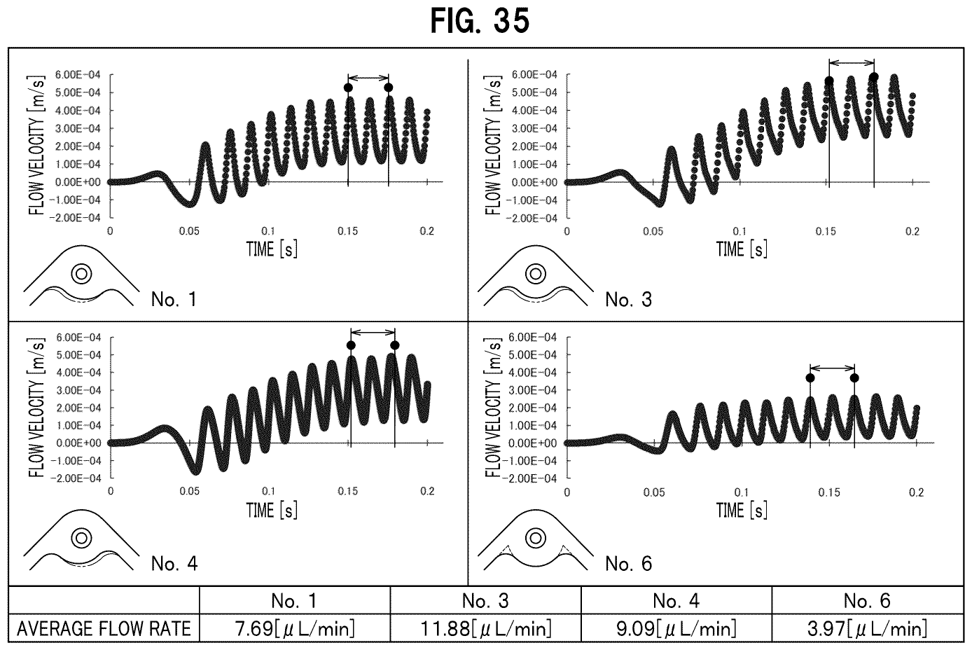

The model was constructed by setting the pump portion (liquid delivery section 3) as a moving mesh (hatched portion in the center figure in the drawing) and the flow path as a stationary mesh. The mesh shape was a tetrahedron, and the element size was fine (Min: 0.0288 mm, Max: 0.152 mm). The physical model was laminar flow, and the material was water. The results are shown in FIG. 35. FIG. 35 is a graph showing the results of the simulation. The upper left in the figure is No. 1, the upper right is No. 3, the lower left is No. 4, and the lower right is No. 6. The horizontal axis in each graph represents time [s], and the vertical axis represents flow velocity [m/s].

As shown in FIG. 35, as a result of the simulation, the average flow rates obtained from one rotation cycle of the impeller 20 shown in each graph are 7.69 μL/min for No. 1, 11.88 μL/min for No. 3, 9.09 μL/min for No. 4, and 3.97 μL/min for No. 6.

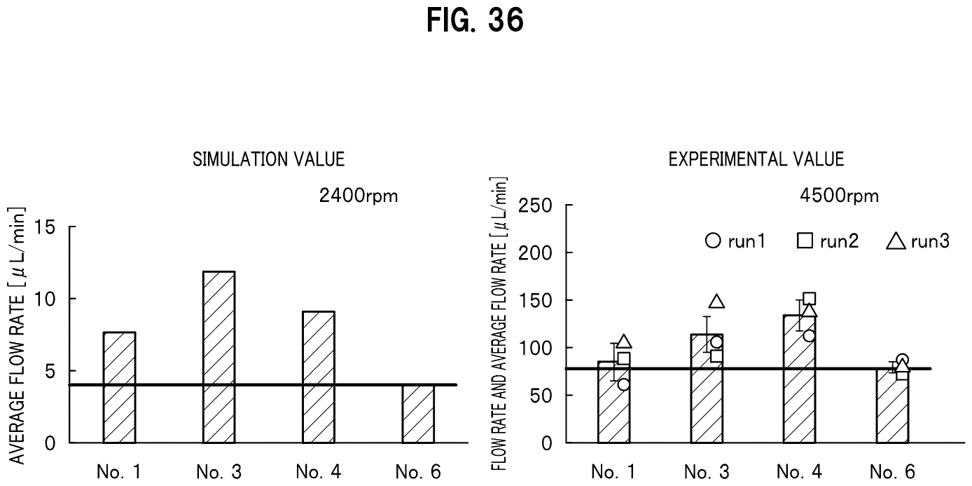

FIG. 36 is an explanatory diagram showing a graph (left graph) showing the average flow rates described in the table shown in FIG. 35 (experimental results in the simulation) in a bar graph, and a graph (right graph) extracting the experimental results of Nos. 1, 3, 4, and 6 (experimental results with actual devices) from the graph shown in FIG. 33. In the graph on the right, ∘ indicates the flow rate of run 1, □ indicates the flow rate of run 2, and Δ indicates the flow rate of run 3, and the bar graph indicates the average flow rate. The error bar indicates the variation (inter-experimental variation) in the flow rate of the three experiments (runs 1 to 3) performed using the same device, that is, the standard deviation.

The simulation values in the left graph in FIG. 36 were obtained at a rotation speed of the impeller 20 of 2400 rpm. The experimental values in the right graph were obtained at a rotation speed of the impeller 20 of 4500 rpm. In either case, Nos. 1, 3, and 4 in which at least one of the formation dimension FD3 on the liquid inflow side and the formation dimension FD4 on the liquid outflow side is longer than that of No. 6 resulted in a larger liquid flow rate than that of No. 6.

### Example 4

### Fourth Example

### (Study on the Influence of the Width Dimension of the Return Flow Path 13)

In [Fourth Example], the influence of changing the width dimension of the return flow path 13 on the liquid flow rate due to “flow path resistance” was verified.

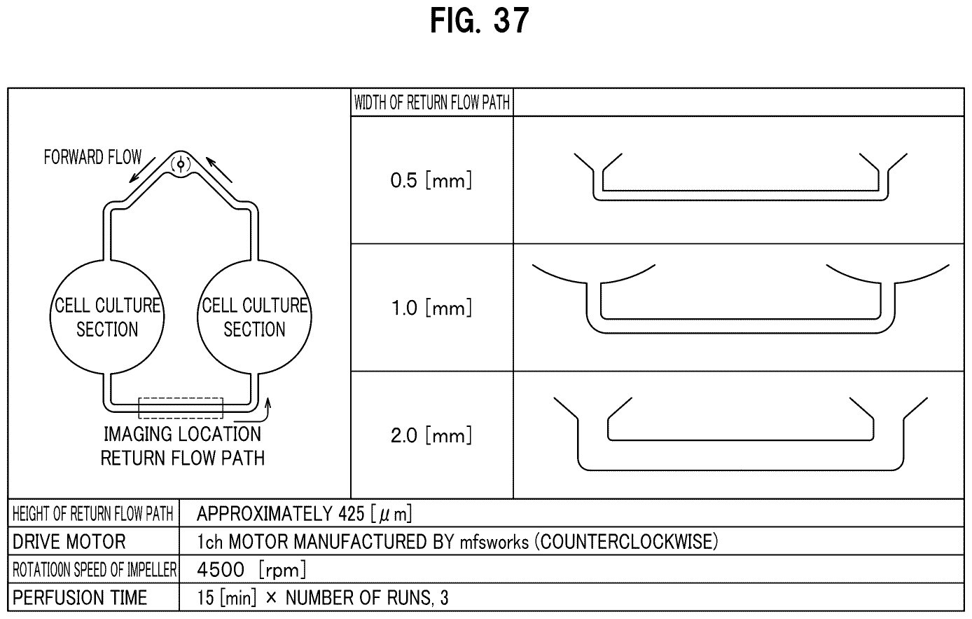

Similarly to [First Example], three device bodies 10 made of PDMS and having a rectangular shape in top-down view, which are formed in a block shape, were prepared. [Fourth Example] is such that the PDMS device body 10 is formed into the shape shown on the left side in FIG. 37 by soft lithography instead of cutting with a three-dimensional plotter. At this time, the device bodies 10 were prepared with three width dimensions of the return flow path 13 of 0.5 mm, 1.0 mm, and 2.0 mm shown on the right side in the figure. FIG. 37 is an explanatory diagram showing and explaining the conditions, etc., of the experiment to examine the influence of the width dimension of the return flow path 13.

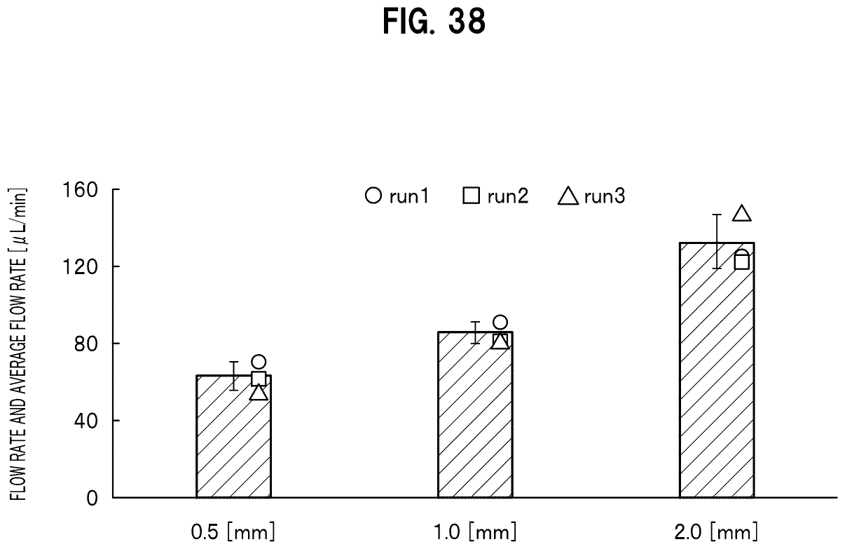

The experimental conditions are as shown in the table at the bottom of FIG. 37; the depth of the return flow path 13 was about 425 μm, the drive motor 31 used was a 1ch motor manufactured by mfsworks, and the impeller 20 was rotated counterclockwise. The rotation speed of the impeller 20 was 4500 rpm. The perfusion time was 15 min, and the measurement was performed three times (number of runs: 3). The liquid flow rate was measured (imaged) in the “Return Flow Path” shown in FIG. 40. The results are shown in FIG. 38.

FIG. 38 is a graph showing the results of verifying the influence of the width dimension of the return flow path 13. In the figure, the vertical axis represents the flow rate and the average flow rate [μL/min]. ∘ indicates the flow rate of run 1, □ indicates the flow rate of run 2, and Δ indicates the flow rate of run 3, and the bar graph indicates the average flow rate. The error bar indicates the variation (inter-experimental variation) in the flow rate of the three experiments (runs 1 to 3) performed using the same device, that is, the standard deviation.

As shown in FIG. 38, the liquid flow rate increased as the width dimension of the return flow path 13 increased. From this, it was confirmed that the liquid flow rate can be increased by increasing the width dimension of the return flow path 13 and lowering the flow path resistance. Further, it was confirmed that the liquid flow rate can be decreased by decreasing the width dimension of the return flow path 13 and increasing the flow path resistance.

### REFERENCE SIGNS LIST

100 Liquid delivery device
1, 1A to 1F Liquid flow structure
2 Liquid flow section
3 Liquid delivery section
4 Loop-shaped flow path
5 First reservoir
5a Bottom surface
5b Inner wall surface
6 Second reservoir
6a Bottom surface
6b Inner wall surface
7 Liquid delivery chamber
7a Inner peripheral surface
7al First inner peripheral surface
7a2 Second inner peripheral surface
7a3 Third inner peripheral surface
7a4 Fourth inner peripheral surface
7b Support shaft
7c Outer periphery
7d Center point
7e First wall portion
7f Second wall portion
8 Liquid delivery rotation section
9 Support portion
10 Device body
10a Main surface
11 First flow path
11a One end (one end of first flow path 11)
11b Bent portion
11c Connection end
11d Inner side surface
12 Second flow path
12a One end (one end of second flow path 12)
12b Bent portion
12c Connection end
12d Inner side surface
13 Return flow path
13a One end (one end of return flow path 13)
13b Other end (other end of return flow path 13)
14 Liquid inlet/outlet
14a End (end of liquid inlet/outlet 14)
15 Liquid inlet/outlet
15a End (end of liquid inlet/outlet 15)
20 Impeller
21 Annular portion
21a Inner circle
21b Bearing portion
22 Blade portion
22a Tip
23 Rotation trajectory
24 Gap
30 Drive device
30a Mounting surface
31 Drive motor
32 Positioning portion
A1 Area corresponding to first quadrant
A2 Area corresponding to second quadrant
A3 Area corresponding to third quadrant
A4 Area corresponding to fourth quadrant
A5 Semicircular area
C1 Central axis
C2 Rotation center (rotation center of annular portion 21)
C3 Rotation center (rotation center of drive motor 31)
D1 Rotation direction (counterclockwise direction)
D2 Rotation direction (clockwise direction)
F1 Forward flow
F2 Reverse flow
IL, ILa Virtual line
L1, L2 Tangent
RL1 First reference line
RL2 Second reference line
RL3 Third reference line
RL4 Fourth reference line
RL5 Fifth reference line
PT First connection portion
P2 Second connection portion
P3, P3a Third connection portion
P4, P4a Fourth connection portion
P5 Point bisecting length of first wall portion 7e
P6 Intersection (intersection between second inner peripheral surface 7a2 and first reference line RL1)
S1 Center
S2 Upper right
S3 Upper left
S4 Lower left
S5 Lower right
S6 Top
S7 Center
S8 Bottom
S9 Upper left
S10 Upper right
V1, V2 Velocity vector
W1 to W3 Width

1. A liquid delivery device comprising a liquid delivery section and a liquid delivery rotation section, wherein
the liquid delivery section includes:
a liquid delivery chamber in which liquid flows in and out; and
a linear first flow path and a linear second flow path that allow the liquid to flow to and from the liquid delivery chamber in top-down view of the liquid delivery chamber,
the liquid delivery rotation section includes:
a support shaft that is arranged to project in a center of the liquid delivery chamber;
an impeller including an annular portion rotatably supported by the support shaft, and a blade portion provided in the annular portion and causing the liquid in the liquid delivery chamber to flow out of the first flow path or the second flow path, the impeller being made of a material containing a magnetic material; and
a drive motor that is arranged outside the liquid delivery chamber and rotates the impeller by a magnetic field,
a support portion configured by an outer periphery of the support shaft and an inner circle of the annular portion has a gap for the annular portion to be rotatable, and
the drive motor is arranged so that the impeller rotates with the gap being biased in a contact range between the outer periphery of the support shaft and the inner circle of the annular portion.

2. The liquid delivery device according to claim 1, wherein
in the top-down view, the first flow path and the second flow path are in any one positional relationship of line symmetry, asymmetry, and point symmetry with respect to the liquid delivery chamber with reference to the support shaft.

3. The liquid delivery device according to claim 1, wherein
the liquid delivery chamber is circular in the top-down view,
a length of a first wall portion, which is formed by a first connection portion connected to the first flow path, a second connection portion connected to the second flow path, and a circular inner peripheral surface between the first connection portion and the second connection portion in the top-down view of the liquid delivery chamber is shorter than a length of a second wall portion, which is formed by a third connection portion connected to the first flow path, a fourth connection portion connected to the second flow path, and a circular inner peripheral surface between the third connection portion and the fourth connection portion,
the first flow path and the second flow path are provided so that an inflow direction of the liquid flowing into the liquid delivery chamber and an outflow direction of the liquid flowing out of the liquid delivery chamber are aligned continuously by an arc of the first wall portion, and
the drive motor is arranged so that an area is bisected by a second reference line passing through a center point of the support shaft and orthogonal to a first reference line passing through the center point and a point bisecting a length of the first wall portion, a rotation center of the impeller is located in a semicircular area including the first wall portion, and the impeller rotates with the gap being biased.

4. The liquid delivery device according to claim 1, wherein
the liquid delivery chamber is circular in the top-down view,
a length of a first wall portion, which is formed by a first connection portion connected to the first flow path, a second connection portion connected to the second flow path, and a circular inner peripheral surface between the first connection portion and the second connection portion in the top-down view of the liquid delivery chamber is shorter than a length of a second wall portion, which is formed by a third connection portion connected to the first flow path, a fourth connection portion connected to the second flow path, and a circular inner peripheral surface between the third connection portion and the fourth connection portion,
the first flow path and the second flow path are formed on the same straight line via the liquid delivery chamber,
in a concentric circle centered on a center point of the support shaft, the liquid delivery chamber includes the first flow path at a position corresponding to a first quadrant with respect to a third reference line passing through the center point and parallel to an inflow direction of the liquid to the liquid delivery chamber and a fourth reference line orthogonal to the third reference line and passing through the center point, and includes the second flow path at a position corresponding to a second quadrant with respect to the third reference line and the fourth reference line, and
the drive motor is arranged so that a rotation center of the impeller is located in an area corresponding to the first quadrant or an area corresponding to a third quadrant with respect to the third reference line and the fourth reference line, and the impeller rotates with the gap being biased.

5. The liquid delivery device according to claim 1, wherein
the liquid delivery chamber is circular in the top-down view,
a length of a first wall portion, which is formed by a first connection portion connected to the first flow path, a second connection portion connected to the second flow path, and a circular inner peripheral surface between the first connection portion and the second connection portion in the top-down view of the liquid delivery chamber is shorter than a length of a second wall portion, which is formed by a third connection portion connected to the first flow path, a fourth connection portion connected to the second flow path, and a circular inner peripheral surface between the third connection portion and the fourth connection portion,
a formation direction of the first flow path with respect to the liquid delivery chamber and a formation direction of the second flow path with respect to the liquid delivery chamber are parallel, but the first flow path and the second flow path are formed to be offset with respect to each other via the liquid delivery chamber,
the impeller rotates in the same direction as an outflow direction of the liquid flowing from the first flow path to the second flow path,
in a concentric circle centered on a center point of the support shaft, the liquid delivery chamber includes the first flow path at a position including a boundary line between a first quadrant and a fourth quadrant with respect to a third reference line passing through the center point and parallel to an inflow direction of the liquid to the liquid delivery chamber and a fourth reference line orthogonal to the third reference line and passing through the center point, and includes the second flow path at a position corresponding to a second quadrant with respect to the third reference line and the fourth reference line, and
the drive motor is arranged so that a rotation center of the impeller is located in an area corresponding to the first quadrant or an area corresponding to a third quadrant with respect to the third reference line and the fourth reference line, and the impeller rotates with the gap being biased.

6. The liquid delivery device according to claim 1, wherein
the liquid delivery chamber is circular in the top-down view,
a length of a first wall portion, which is formed by a first connection portion connected to the first flow path, a second connection portion connected to the second flow path, and a circular inner peripheral surface between the first connection portion and the second connection portion in the top-down view of the liquid delivery chamber is shorter than a length of a second wall portion, which is formed by a third connection portion connected to the first flow path, a fourth connection portion connected to the second flow path, and a circular inner peripheral surface between the third connection portion and the fourth connection portion,
a formation direction of the first flow path with respect to the liquid delivery chamber and a formation direction of the second flow path with respect to the liquid delivery chamber are parallel, but the first flow path and the second flow path are formed to be offset with respect to each other via the liquid delivery chamber,
the impeller rotates in the same direction as an outflow direction of the liquid flowing from the second flow path to the first flow path,
in a concentric circle centered on a center point of the support shaft, the liquid delivery chamber includes the first flow path at a position including a boundary line between a first quadrant and a fourth quadrant with respect to a third reference line passing through the center point and parallel to an inflow direction of the liquid to the liquid delivery chamber and a fourth reference line orthogonal to the third reference line and passing through the center point, and includes the second flow path at a position corresponding to a second quadrant with respect to the third reference line and the fourth reference line, and
the drive motor is arranged so that a rotation center of the impeller is located in an area corresponding to the first quadrant, an area corresponding to a second quadrant, or an area corresponding to a fourth quadrant, and the impeller rotates with the gap being biased.

7. The liquid delivery device according to claim 1, wherein
the liquid delivery chamber is circular in the top-down view,
the liquid delivery chamber has a first wall portion and a second wall portion,
the first wall portion is formed by a first connection portion connected to the first flow path, a second connection portion connected to the second flow path, and a first inner peripheral surface forming a circular arc between the first connection portion and the second connection portion in the top-down view,
the second wall portion is formed by a third connection portion connected to the first flow path, a fourth connection portion connected to the second flow path, a second inner peripheral surface forming a circular arc between the third connection portion and the fourth connection portion, a third inner peripheral surface having an arc shape and a curvature opposite to that of the second inner peripheral surface between the third connection portion and the second inner peripheral surface, and a fourth inner peripheral surface having an arc shape and a curvature opposite to that of the second inner peripheral surface between the fourth connection portion and the second inner peripheral surface,
the first flow path and the second flow path are provided so that an inflow direction of the liquid flowing into the liquid delivery chamber and an outflow direction of the liquid flowing out of the liquid delivery chamber are aligned continuously by an arc of the first wall portion, and
the drive motor is arranged so that an area is bisected by a second reference line passing through a center point of the support shaft and orthogonal to a first reference line passing through the center point and a point bisecting a length of the first wall portion, a rotation center of the impeller is located in a semicircular area including the first wall portion, and the impeller rotates with the gap being biased.

8. The liquid delivery device according to claim 7, wherein
taking, as a reference, a fifth reference line that passes through an intersection between the second inner peripheral surface and the first reference line and is parallel to the second reference line, formation dimensions of the third inner peripheral surface and the fourth inner peripheral surface, measured perpendicular to the fifth reference line at longest points thereof, are both the same.

9. The liquid delivery device according to claim 7, wherein
taking, as a reference, a fifth reference line that passes through an intersection between the second inner peripheral surface and the first reference line and is parallel to the second reference line, formation dimensions of the third inner peripheral surface and the fourth inner peripheral surface, measured perpendicular to the fifth reference line at longest points thereof, are different from each other.

10. The liquid delivery device according to claim 1, wherein
in the liquid delivery chamber, when the liquid is delivered by rotation of the impeller, a flow rate of the liquid is adjusted by a rotation speed of the impeller.

11. The liquid delivery device according to claim 1, wherein
a liquid delivery direction of the liquid is adjusted by a rotation direction of the impeller.

12. The liquid delivery device according to claim 1, further comprising:
a first reservoir communicating with the first flow path;
a second reservoir communicating with the second flow path; and
a return flow path connected to the first reservoir and the second reservoir, wherein
the first flow path, the second flow path, and the return flow path form a loop-shaped flow path,
a depth dimension of the loop-shaped flow path and a depth dimension of the liquid delivery chamber are the same, and
a flow rate of the liquid is adjusted by the depth dimension.

13. The liquid delivery device according to claim 12, wherein
a flow rate of the liquid is adjusted by a width dimension of the return flow path.
